(19) United States
(12) Patent Application Publication
(10) Pub. No.: US 2024/0091242 A1
(43) Pub. Date: Mar. 21, 2024

(54) LIPIDS THAT REDUCE LUNG DAMAGE, IMPROVE PULMONARY FUNCTION AND DECREASE PRO-INFLAMMATORY CYTOKINES

(71) Applicant: Signpath Pharma, Inc., Sandy, UT (US)

(72) Inventors: Peter P. Sordillo, New York, NY (US); Dan Salvail, Sherbrooke (CA); Sebastien M. Labbe, Sherbrooke (CA)

(21) Appl. No.: 18/260,012

(22) PCT Filed: Jan. 21, 2022
(86) PCT No.: PCT/US22/13283

§ 371 Date: Jun. 29, 2023

Related U.S. Application Data
(60) Provisional application No. 63/143,511, filed on Jan. 29, 2021.

(51) Int. Cl. A61K 31/683; A61K 45/06; A61P 11/00; A61P 29/00

(52) U.S. Cl. CPC A61K 31/683; A61K 45/06; A61P 11/00; A61P 29/00

(57) ABSTRACT

A method of treating a disease or pathology caused by an increase in levels of inflammatory cytokines comprising providing the subject with a compound of Formula I, (I) wherein, R¹ is a C₁-C₂₀ branched or unbranched hydrocarbon; R³ is (II); or R⁴ is H or a pharmaceutically acceptable cation; R⁵ is a C₁-C₁₀ branched or unbranched hydrocarbon or optionally substituted with one or more groups; R⁶ is a C₁-C₁₀ branched or unbranched hydrocarbon or optionally substituted; R7 is a C₀-C₂₀ branched or unbranched hydrocarbon; R⁸ is H or a C₀-C₂₀ branched or unbranched hydrocarbon; X is a direct linkage, CH₂, O or NH; Y is a direct linkage, CH₂, O or NH; and, each stereogenic center is independently R, S or racemic.

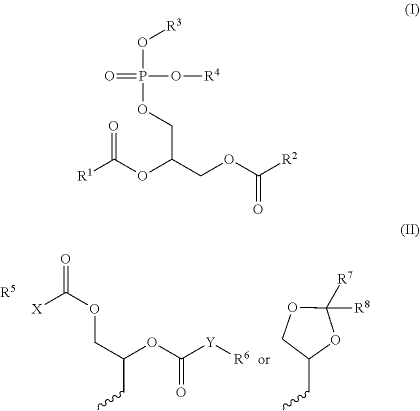

### CROSS-REFERENCE TO RELATED APPLICATIONS

This PCT International Application claims priority to U.S. Provisional Patent Application Ser. No. 63/143,511 filed Jan. 29, 2021, the contents of which is incorporated by reference herein in its entirety.

### TECHNICAL FIELD OF THE INVENTION

The present invention relates in general to the field of compositions and methods to reduce pro-inflammatory cytokines, and treatment of disease symptoms that result from pro-inflammatory cytokines, including lung damage, impaired pulmonary function and/or acute respiratory distress syndrome (ARDS).

### STATEMENT OF FEDERALLY FUNDED RESEARCH

None.

### BACKGROUND OF THE INVENTION

There are many disease states and pathologies that involve pro-inflammatory cytokines, including sepsis, Alzheimer's disease, traumatic brain injury, Ebola, arthritis, and other situations where reduction in pro-inflammatory cytokines can be beneficial. Without limiting the scope of the invention, its background is described in connection with Acute respiratory distress syndrome (ARDS).

One example of a disease that involves pro-inflammatory cytokines is Acute respiratory distress syndrome (ARDS) is an intense inflammatory process in the lung that results in a high mortality characterized by severe hypoxemia following acute lung injury. Etiologic factors of ARDS include sepsis, pneumonia, acute pancreatitis, chemical or smoke inhalation, aspiration of gastric contents, traumatic shock, chemotherapy toxicity, or viral illnesses including COVID-19.¹⁻⁶ Hypoxemia secondary to cardiogenic pulmonary edema is excluded in the diagnosis of ARDS. In patients with ARDS, damage to the lung can begin hours or days after the initial insult, with damage to alveoli and major edema in the lungs which are manifested by diffuse infiltrates on chest radiograph. After 7-10 days, damage to the lung can progress to fibrosis.^{7, 8} Patients with ARDS lung damage have a decreased PaO/FiO₂ ratio (partial pressure of oxygen in arterial blood/fraction of oxygen in inspired air), indicating the degree to which their lungs can take in oxygen. Decreasing P_{A}O₂/FiO₂ valves indicate worsening lung damage.

Increases in neutrophils are found prominently in the bronchoalveolar lavage fluid (BALF) of ARDS patients, and these cells are important in the progression of this disease.⁹⁻¹¹ Levels of neutrophils and of neutrophil to lymphocyte ratios are prognostic indicators in these patients^{12, 13}, and neutrophil depletion in animal models may partially reduce lung damage.¹⁴ The neutrophils that migrate to the lungs in response to lung inflammation cause the release of pro-inflammatory cytokines. This sets off a cascade of inflammation which increases lung damage.^{15, 16} The neutrophils are known to stimulate particularly the secretion of interleukins (ILs), known to correlate with the severity of ARDS lung damage.¹⁷ The secreted cytokines, in turn, recruit additional neutrophils to the lung.¹⁸ Yang et al, in a recent review (September 2020), reported that prominent cytokines, including interleukins, tumor necrosis factor-α (TNF-α), interferon γ (IFN-γ), and granulocyte colony-stimulating factor (G-CSF) markedly increase in the lung.¹⁶ Others have also reported that these cytokines are particularly important in causing profound inflammation and severe disease.¹⁹⁻²¹ This data in patients has been confirmed in various animal models. In a murine ARDS model, for example, IL-1β, IL-2, IL-5, IL-6, IL-12, IL-17, vascular endothelial growth factor (VEGF), INF-γ, monocyte chemoattract protein-1 (MCP-1, CCL-2), keratinocytes-derived chemokine (KC, CXCL-1), macrophage inflammatory protein-1α (MIP-1α, CCL-3), and interferon gamma-induced protein 10 (IP-10, CXCL-10), were all significantly elevated after 18 hours.²² Further, it is known that mechanical ventilation, when necessary, will cause further lung damage and further inflammation.^{7, 8, 23} Suppression of inflammation is key to treating this disease.

What is needed are novel compositions and methods for the prevention and treatment of ARDS.

### SUMMARY OF THE INVENTION

In one embodiment, the present invention includes a method of treating a disease or pathology caused by an increase in levels of inflammatory cytokines comprising: 1,2-dimyristoyl-sn-glycero-3-phosphocholine (DMPC), 1,2-Dimyristoyl-sn-glycero-3-phosphoglycerol (DMPG), or DMPC/DMPG, the lysophosphatidylglycerol includes at least one of a lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine or a compound of Formula I,

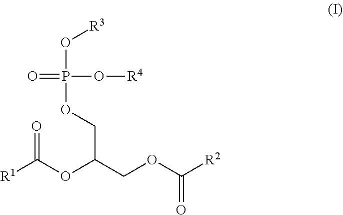

wherein,

R¹ is a C₁-C₂₀ branched or unbranched hydrocarbon possessing 0-10 double bonds, 0-10 triple bonds or a combination of 0-10 double and triple bonds; R² is a C₁-C₂₀ branched or unbranched hydrocarbon possessing 0-10 double bonds, 0-10 triple bonds or a combination of 0-10 double and triple bonds;

R³ is

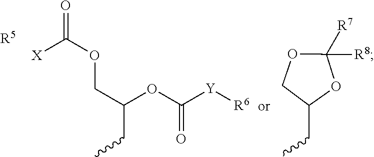

R⁴ is H or a pharmaceutically acceptable cation, wherein incorporation of said pharmaceutically acceptable cation results in a salt; R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁷ is a C₀-C₂₀ branched or unbranched hydrocarbon possessing 0-10 double bonds, 0-10 triple bonds or a combination of 0-10 double and triple bonds; R⁸ is H or a C₀-C₂₀ branched or unbranched hydrocarbon possessing 0-10 double bonds, 0-10 triple bonds or a combination of 0-10 double and triple bonds; X is a direct linkage, CH₂, O or NH; Y is a direct linkage, CH₂, O or NH; and, each stereogenic center is independently R, S or racemic. In one aspect, the disease or pathology caused by an increase in levels of inflammatory cytokines is a pulmonary inflammation, distress or insufficiency. In another aspect, the pulmonary disease includes at least one of bronchopulmonary dysplasia, asthma, chronic obstructive pulmonary disease, bronchitis, chronic or acute bronchoconstriction, acute respiratory distress syndrome, acute lung injury, cytokine storm, or bronchiectasis. In another aspect, the R⁴ is H, Li, Na, K, Mg, Ca, Zn, Cs, ammonium or tetraalkylammonium. In another aspect, the compound is selected from at least one of:

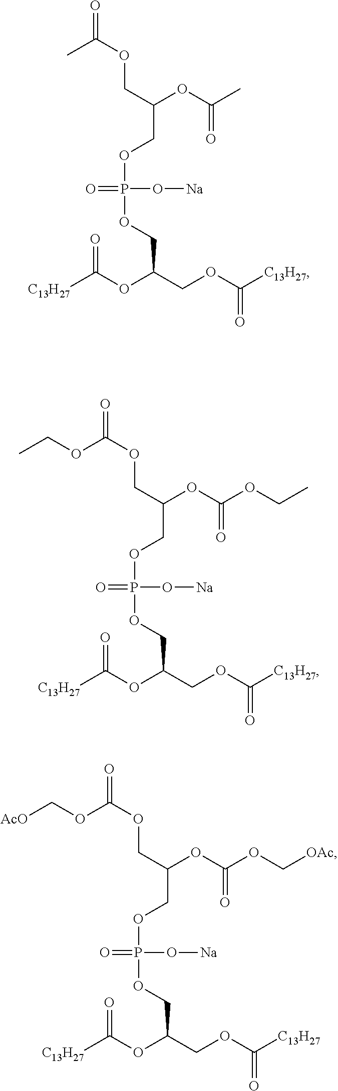

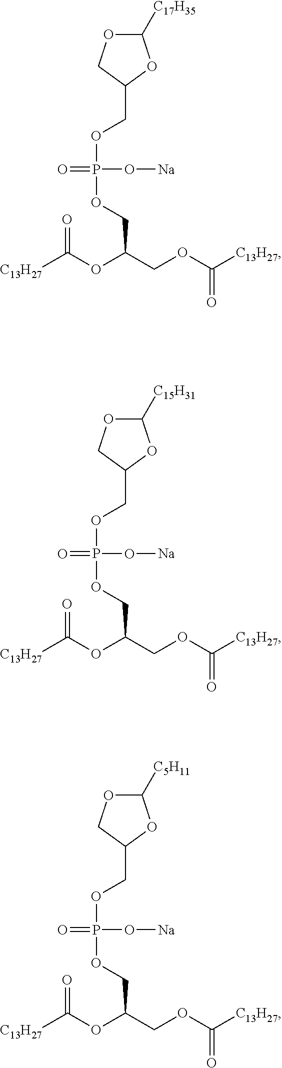

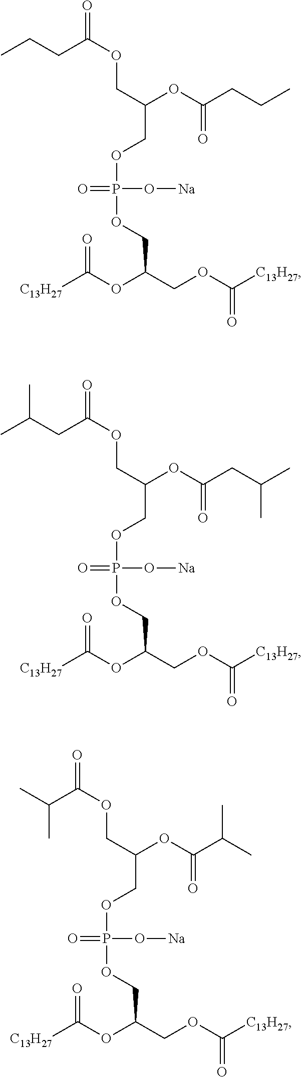

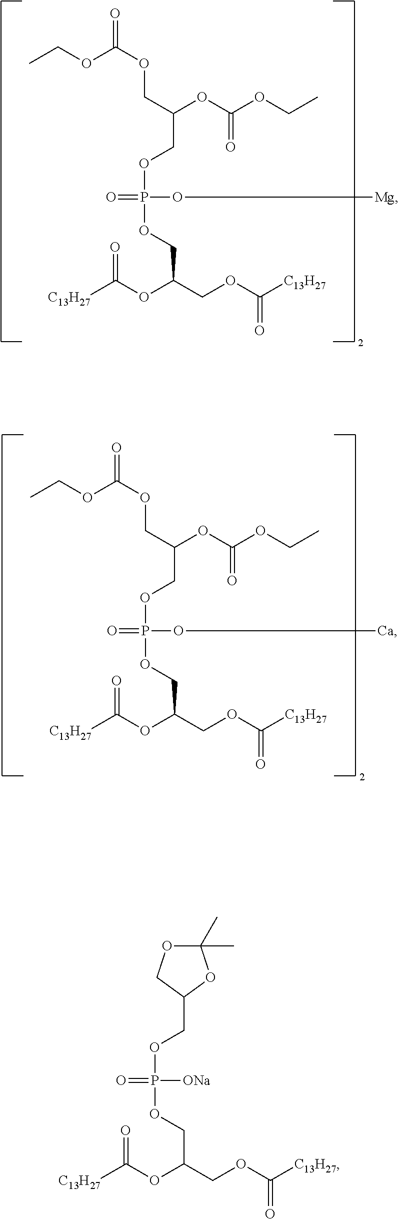

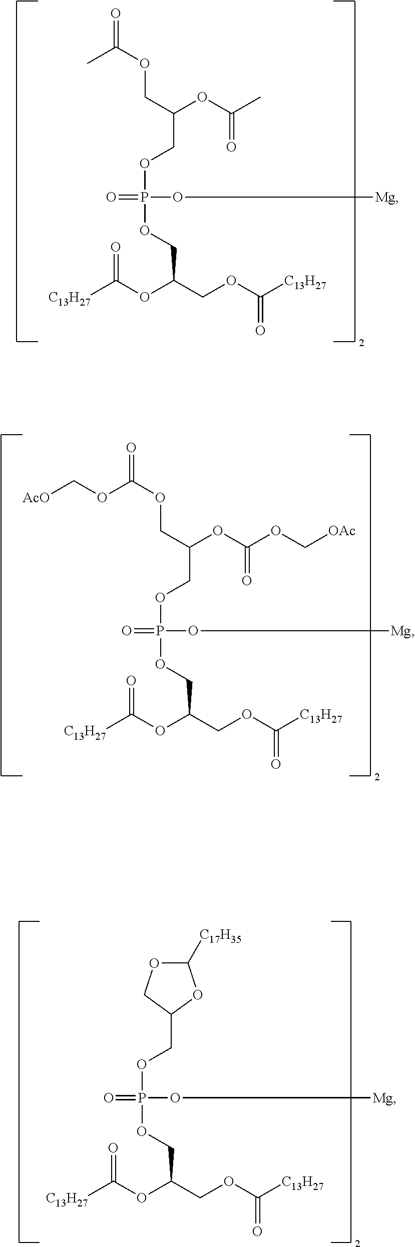

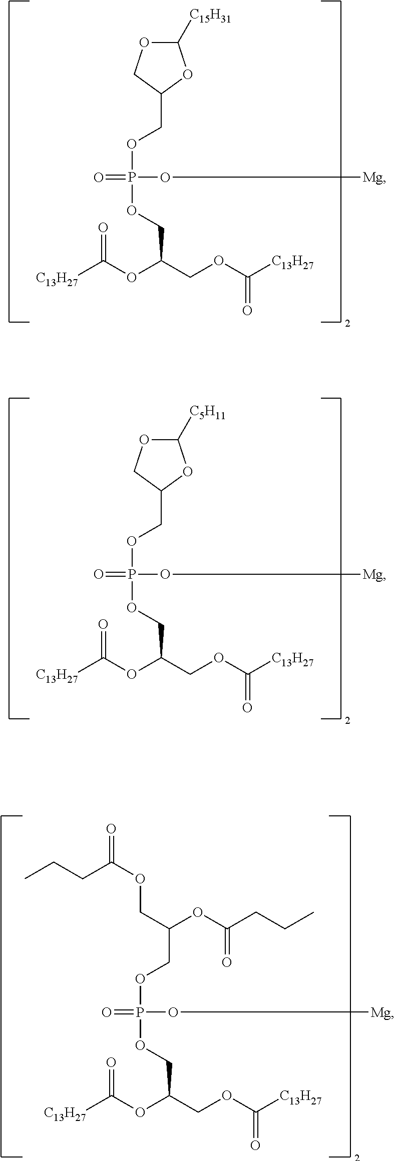

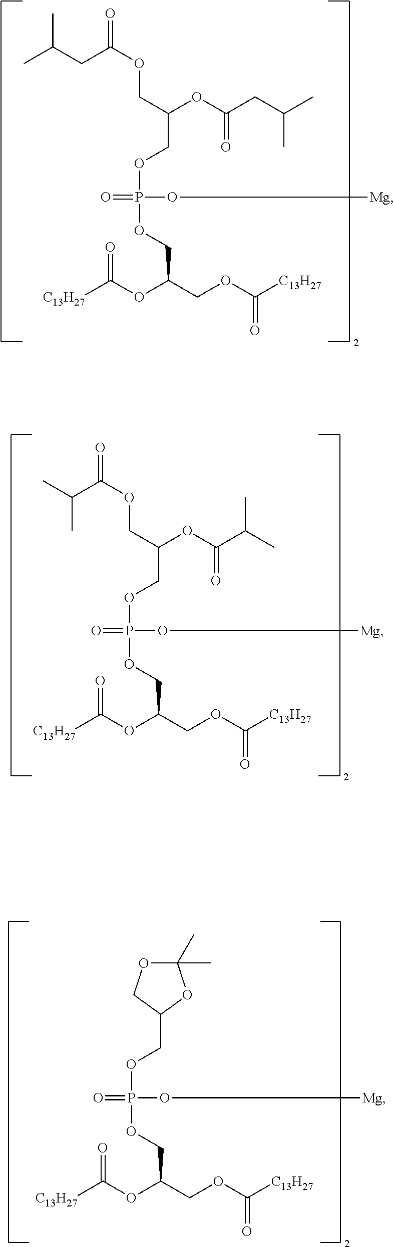

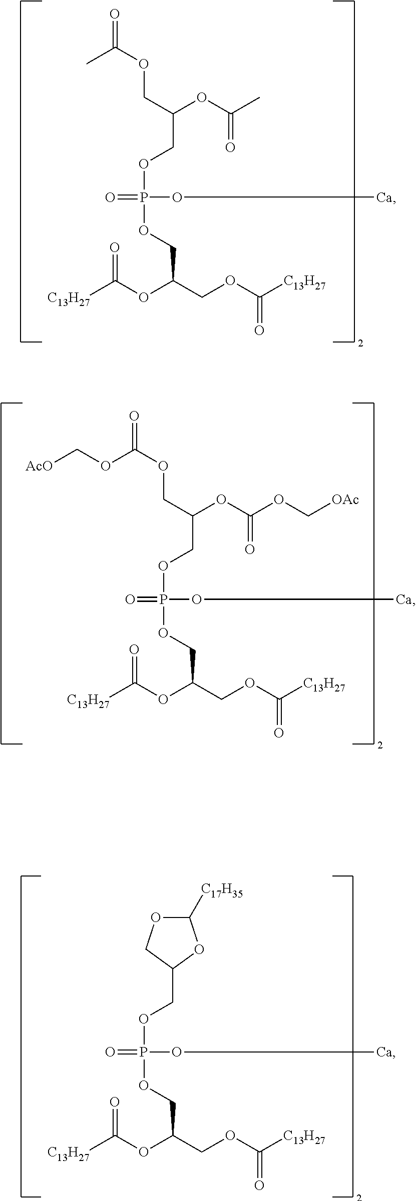

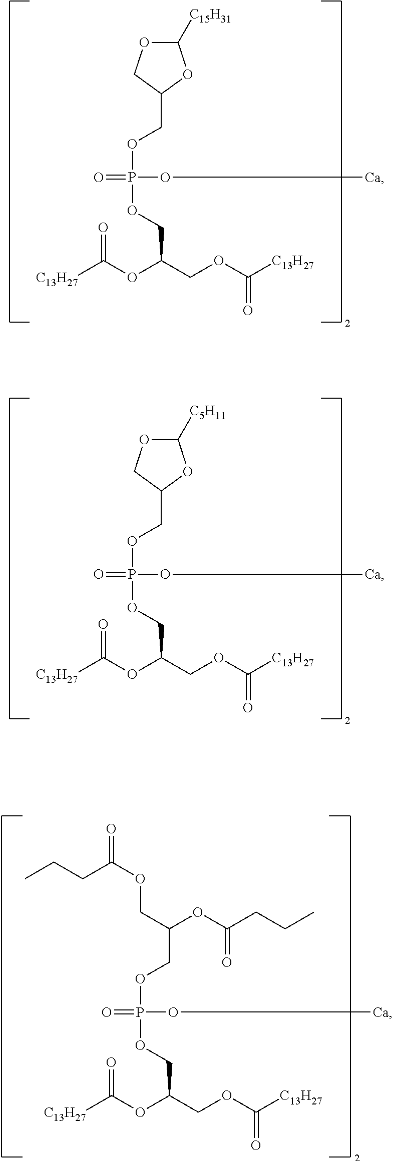

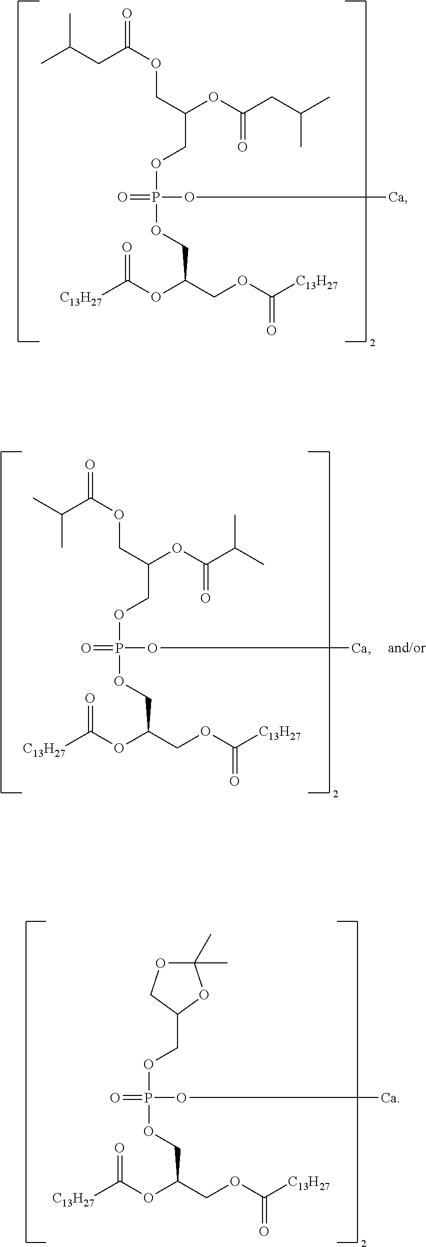

In another aspect, the compound is a single entity, a solvate, a hydrate, a crystal, an amorphous solid, a liquid or an oil. In another aspect, the compound is administered in at least once, once per day, twice per day, three times per day. In another aspect, the compound is administered at 0.1, 1, 2, 3, 4, 5, 6, 7, 89, 10, 15, 20, 25, 30, 40, 50, 60, 75, 80, 90, 100, 125, 150, 175, 200, 255, 250, 300, 400, or 500 mg/Kg. In another aspect, the composition is formulated into a pharmaceutical composition comprising one or more pharmaceutically acceptable excipients, buffers, or salts. In another aspect, the compound is formulated into a pharmaceutical composition adapted for oral, intravenous, nasal, pulmonary, alveolar, enteral, parenteral, or topical administration. In another aspect, the composition is formulated into an aerosol, a nebulizer, or an inhaler. In another aspect, the method further comprises one or more polymers, salts, or buffers. In another aspect, the method further comprises an additional therapeutic agent selected from the group consisting of corticosteroids, bronchodilators, anticholinergics, vasodilators, diuretics, anti-hypertensive agents, acetazolamide, antibiotics, antivirals, immunosuppressive drugs, and surfactants. In another aspect, the subject is a pediatric or adult human or a pediatric or adult animal. In another aspect, the compound is:

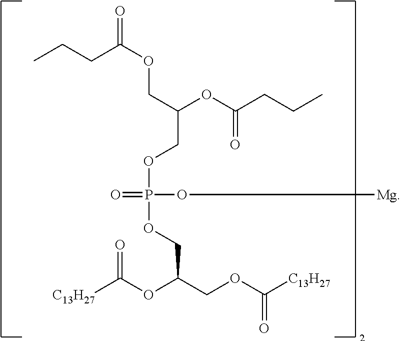

In another embodiment, the present invention includes a method of treating a pulmonary inflammation, distress or insufficiency comprising: 1,2-dimyristoyl-sn-glycero-3-phosphocholine (DMPC), 1,2-Dimyristoyl-sn-glycero-3-phosphoglycerol (DMPG), or DMPC/DMPG, the lysophosphatidylglycerol includes at least one of a lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine or a compound of Formula I,

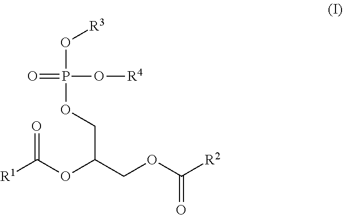

wherein,

R¹ is a C₁-C₂₀ branched or unbranched hydrocarbon possessing 0-10 double bonds, 0-10 triple bonds or a combination of 0-10 double and triple bonds; R² is a C₁-C₂₀ branched or unbranched hydrocarbon possessing 0-10 double bonds, 0-10 triple bonds or a combination of 0-10 double and triple bonds; R³ is

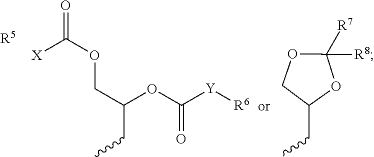

R⁴ is H or a pharmaceutically acceptable cation, wherein incorporation of said pharmaceutically acceptable cation results in a salt; R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁷ is a C₀-C₂₀ branched or unbranched hydrocarbon possessing 0-10 double bonds, 0-10 triple bonds or a combination of 0-10 double and triple bonds; R⁸ is H or a C₀-C₂₀ branched or unbranched hydrocarbon possessing 0-10 double bonds, 0-10 triple bonds or a combination of 0-10 double and triple bonds; X is a direct linkage, CH₂, O or NH; Y is a direct linkage, CH₂, O or NH; and, each stereogenic center is independently R, S or racemic. In one aspect, the pulmonary disease includes at least one of bronchopulmonary dysplasia, asthma, chronic obstructive pulmonary disease, bronchitis, chronic or acute bronchoconstriction, acute respiratory distress syndrome, acute lung injury, cytokine storm, or bronchiectasis. In another aspect, R⁴ is H, Li, Na, K, Mg, Ca, Zn, Cs, ammonium or tetraalkylammonium. In another aspect, the compound is selected from at least one of:

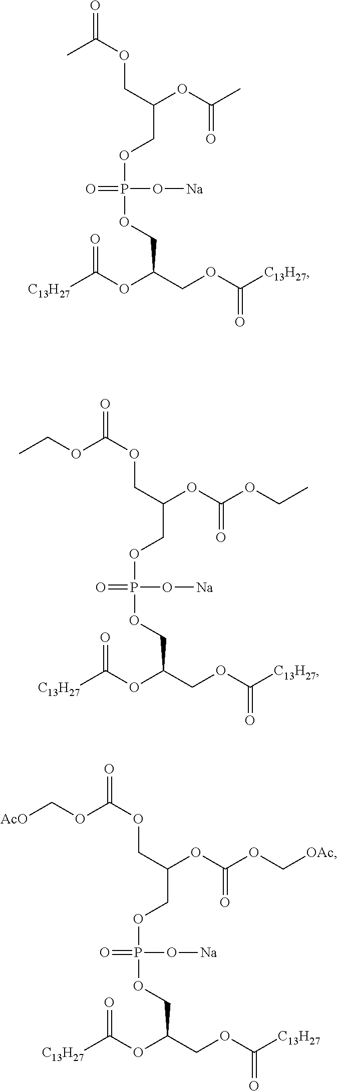

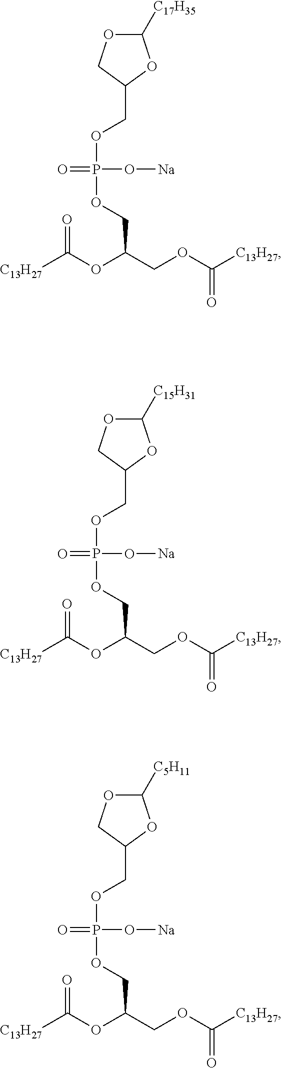

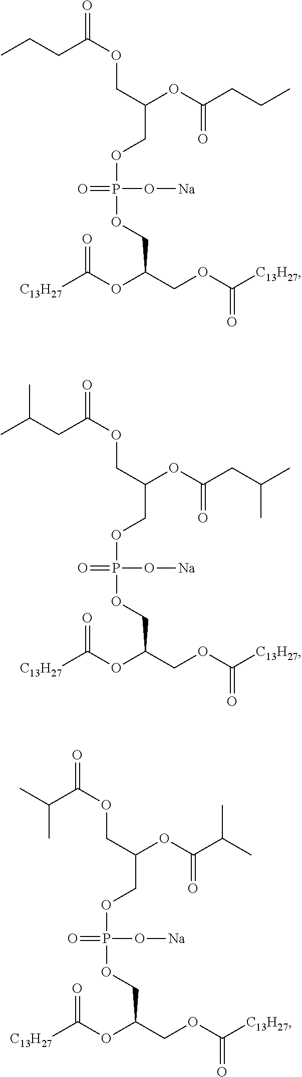

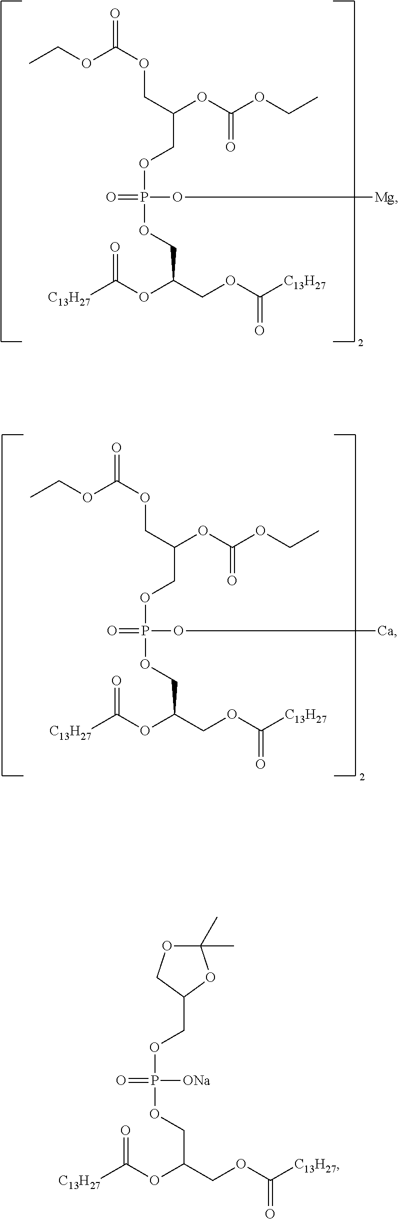

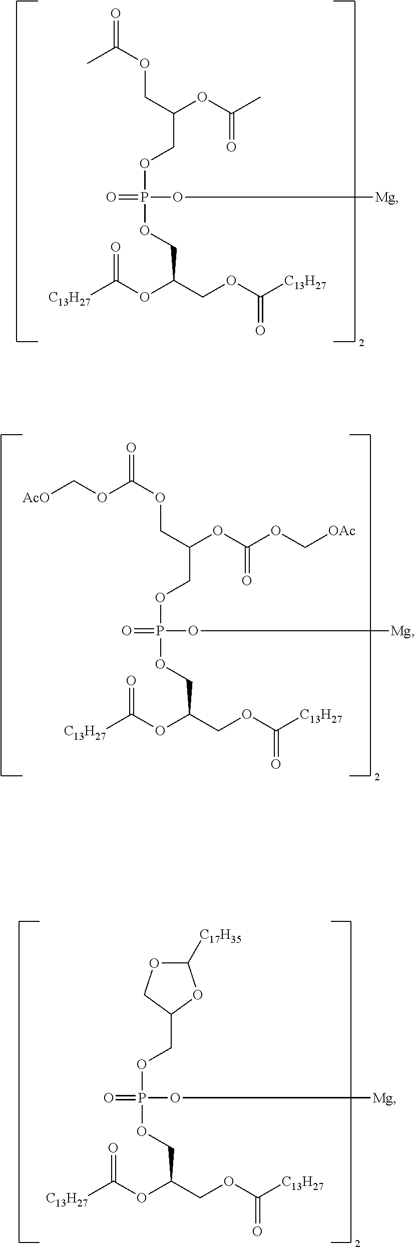

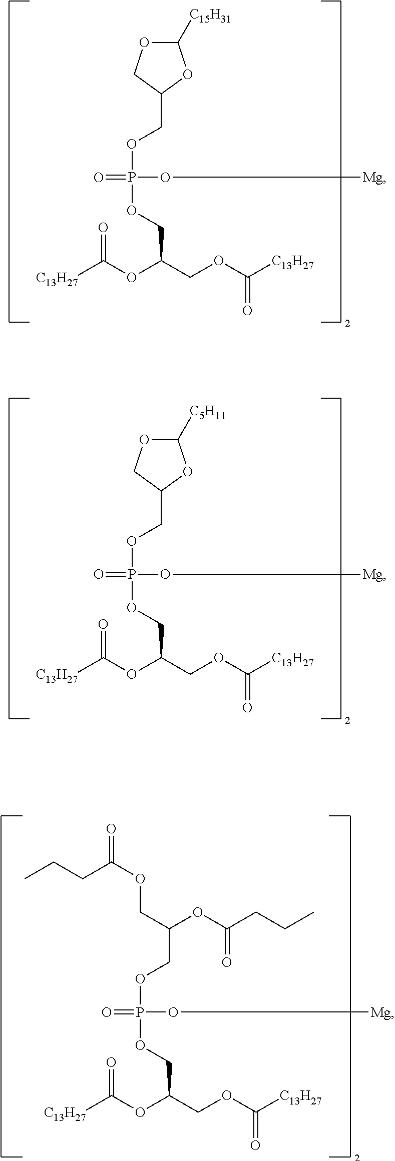

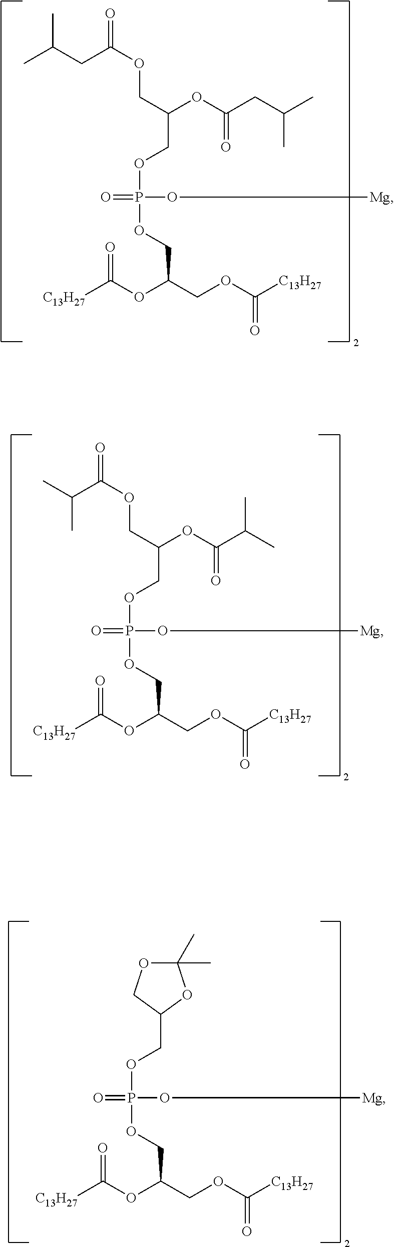

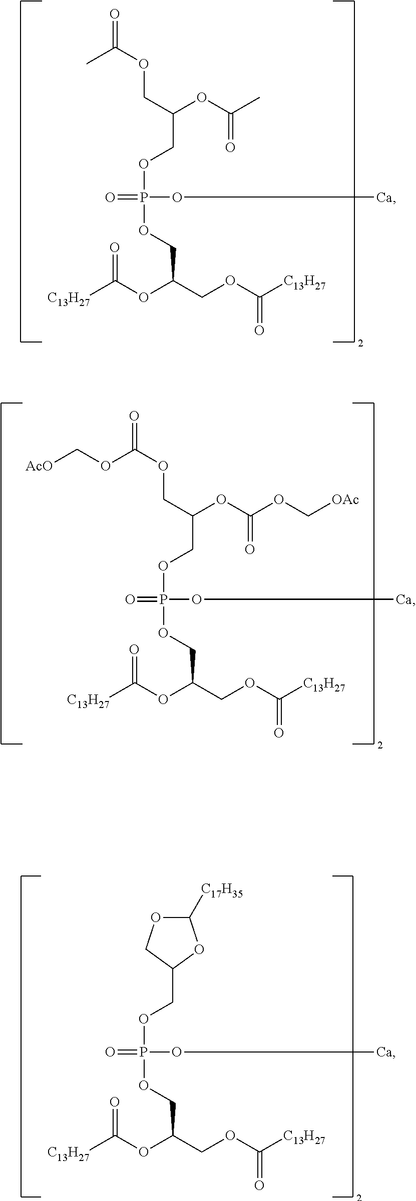

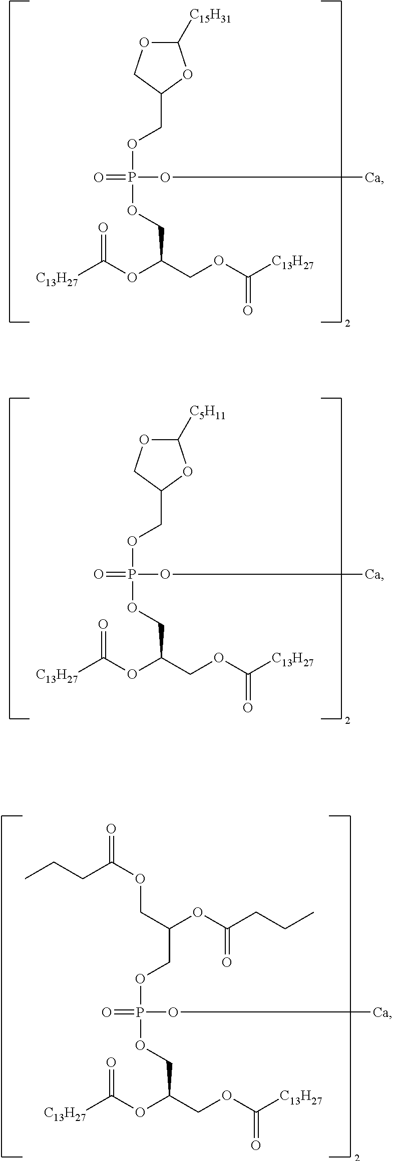

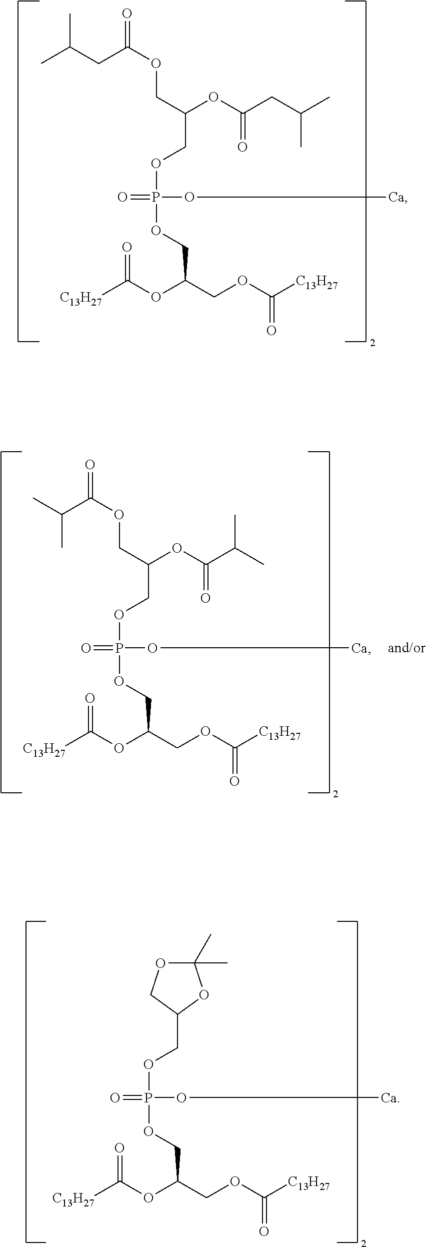

In another aspect, the compound is a single entity, a solvate, a hydrate, a crystal, an amorphous solid, a liquid or an oil. In another aspect, the compound is administered in at least once, once per day, twice per day, three times per day. In another aspect, the compound is administered at 0.1, 1, 2, 3, 4, 5, 6, 7, 89, 10, 15, 20, 25, 30, 40, 50, 60, 75, 80, 90, 100, 125, 150, 175, 200, 255, 250, 300, 400, or 500 mg/Kg. In another aspect, the composition is formulated into a pharmaceutical composition comprising one or more pharmaceutically acceptable excipients, buffers, or salts. In another aspect, the compound is formulated into a pharmaceutical composition adapted for oral, intravenous, nasal, pulmonary, alveolar, enteral, parenteral, or topical administration. In another aspect, the composition is formulated into an aerosol, a nebulizer, or an inhaler. In another aspect, the method further comprises one or more polymers, salts, or buffers. In another aspect, the method further comprises an additional therapeutic agent selected from the group consisting of corticosteroids, bronchodilators, anticholinergics, vasodilators, diuretics, anti-hypertensive agents, acetazolamide, antibiotics, antivirals, immunosuppressive drugs, and surfactants. In another aspect, the subject is a pediatric or adult human or a pediatric or adult animal. In another aspect, the compound is:

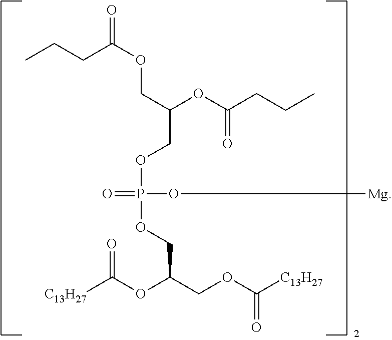

In another embodiment, the present invention includes a method for preventing or treating a pulmonary inflammation, distress or insufficiency comprising: administering to the subject in need thereof a therapeutically effective amount of 1,2-dimyristoyl-sn-glycero-3-phosphocholine (DMPC), 1,2-Dimyristoyl-sn-glycero-3-phosphoglycerol (DMPG), or DMPC/DMPG, the lysophosphatidylglycerol includes at least one of a lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine or a compound of formula (I) or stereoisomer, enantiomer, tautomer or a pharmaceutically acceptable salt thereof:

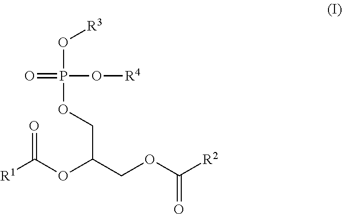

wherein,

R¹ is a C₁-C₂₀ branched or unbranched hydrocarbon possessing 0-10 double bonds, 0-10 triple bonds or a combination of 0-10 double and triple bonds; R² is a C₁-C₂₀ branched or unbranched hydrocarbon possessing 0-10 double bonds, 0-10 triple bonds or a combination of 0-10 double and triple bonds;

R³ is

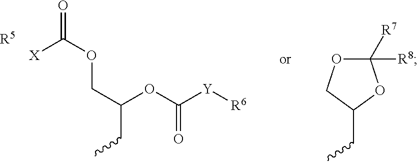

R⁴ is H or a pharmaceutically acceptable cation, wherein incorporation of said pharmaceutically acceptable cation results in a salt; R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁷ is a C₀-C₂₀ branched or unbranched hydrocarbon possessing 0-10 double bonds, 0-10 triple bonds or a combination of 0-10 double and triple bonds; R⁸ is H or a C₀-C₂₀ branched or unbranched hydrocarbon possessing 0-10 double bonds, 0-10 triple bonds or a combination of 0-10 double and triple bonds; X is a direct linkage, CH₂, O or NH; Y is a direct linkage, CH₂, O or NH; and, each stereogenic center is independently R, S or racemic. In one aspect, the pulmonary disease includes at least one of bronchopulmonary dysplasia, asthma, chronic obstructive pulmonary disease, bronchitis, chronic or acute bronchoconstriction, acute respiratory distress syndrome, acute lung injury, cytokine storm, or bronchiectasis. In one aspect, R⁴ is H, Li, Na, K, Mg, Ca, Zn, Cs, ammonium or tetraalkylammonium. In another aspect, the compound is selected from at least one of:

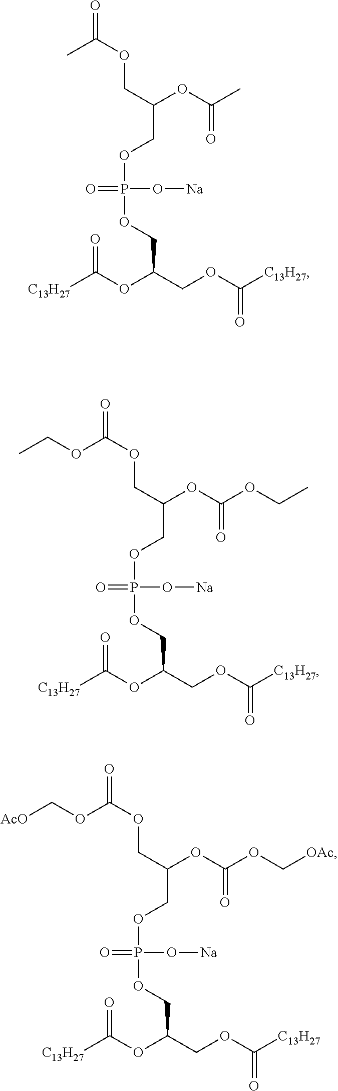

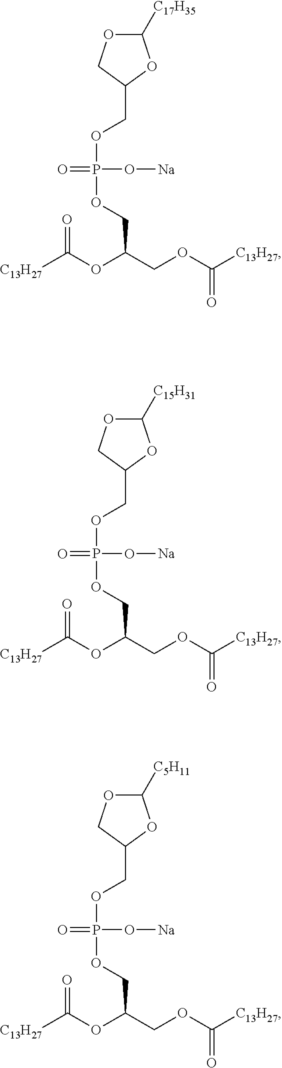

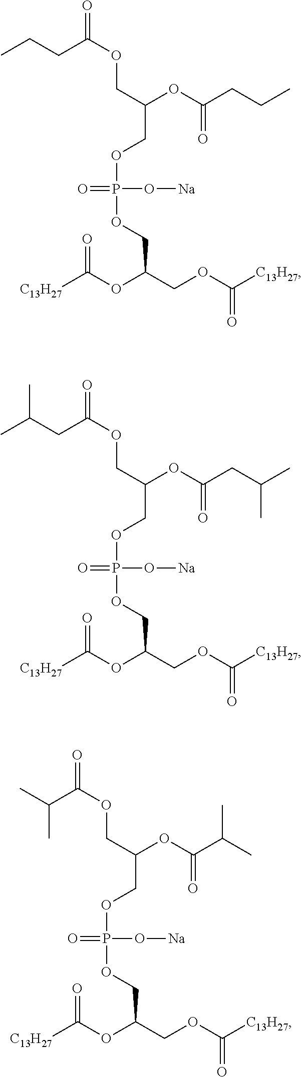

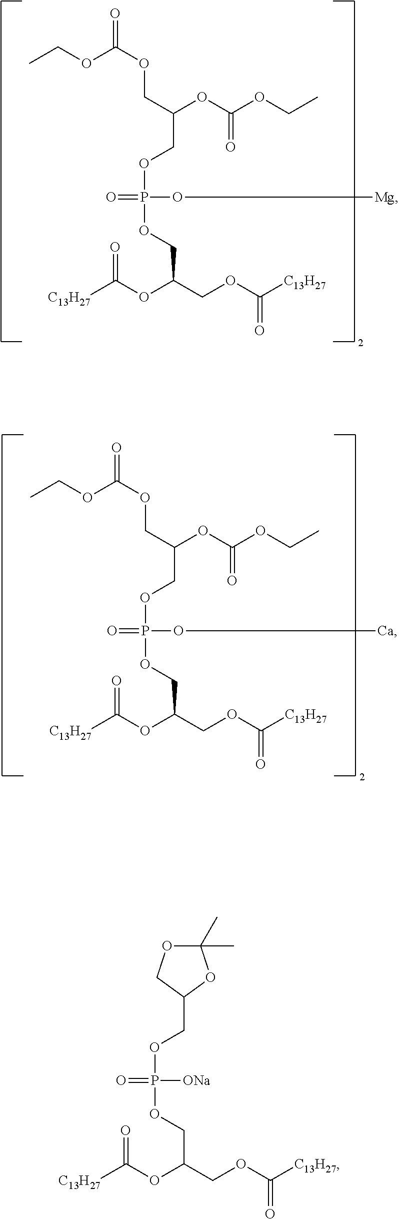

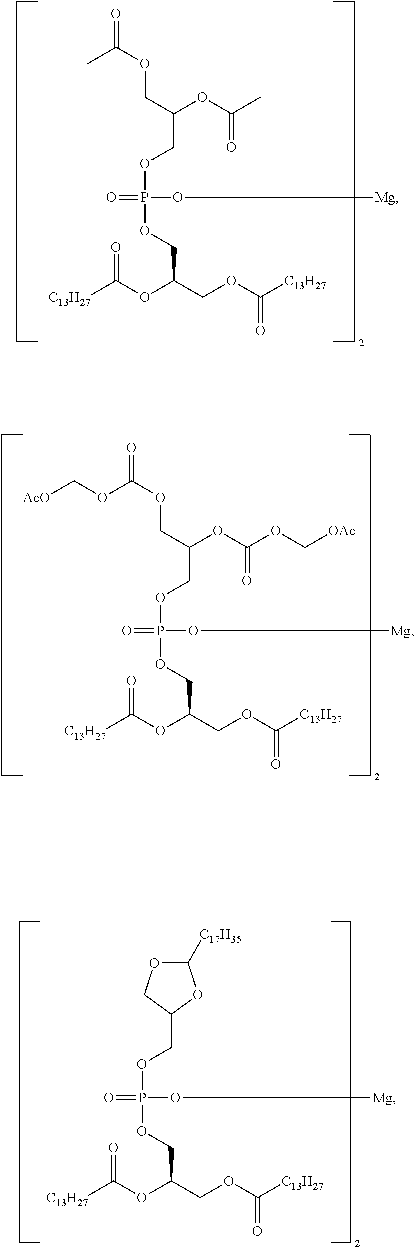

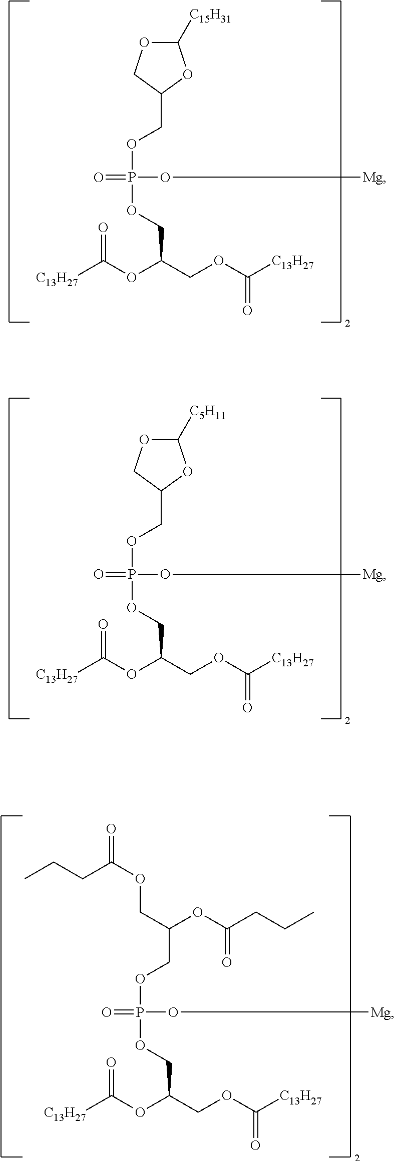

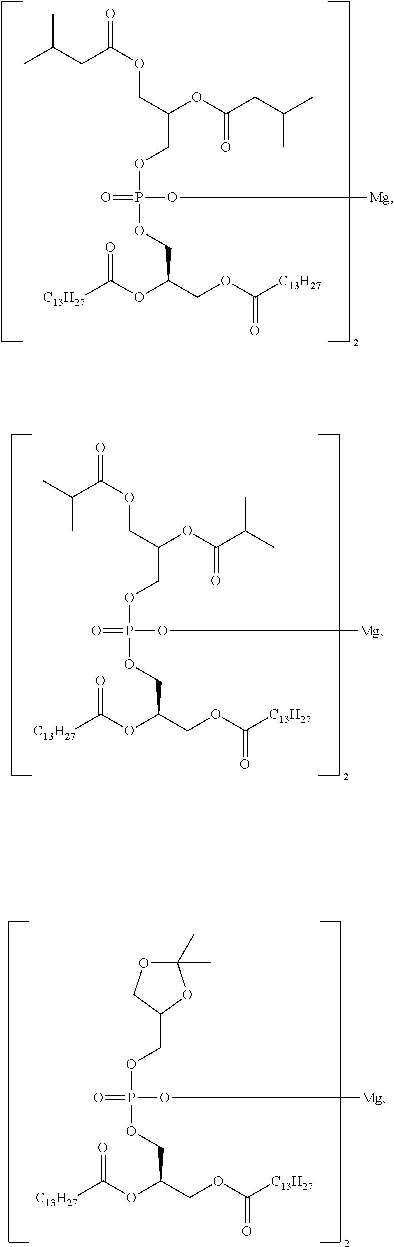

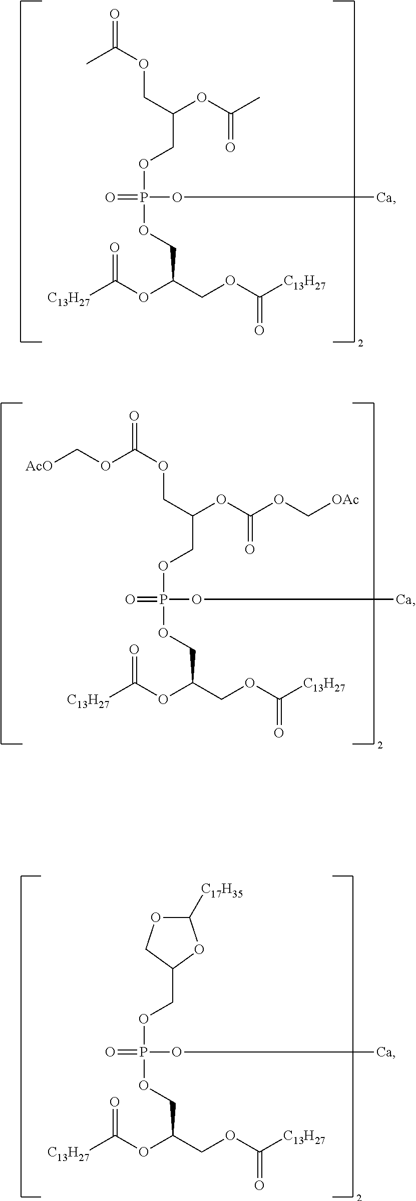

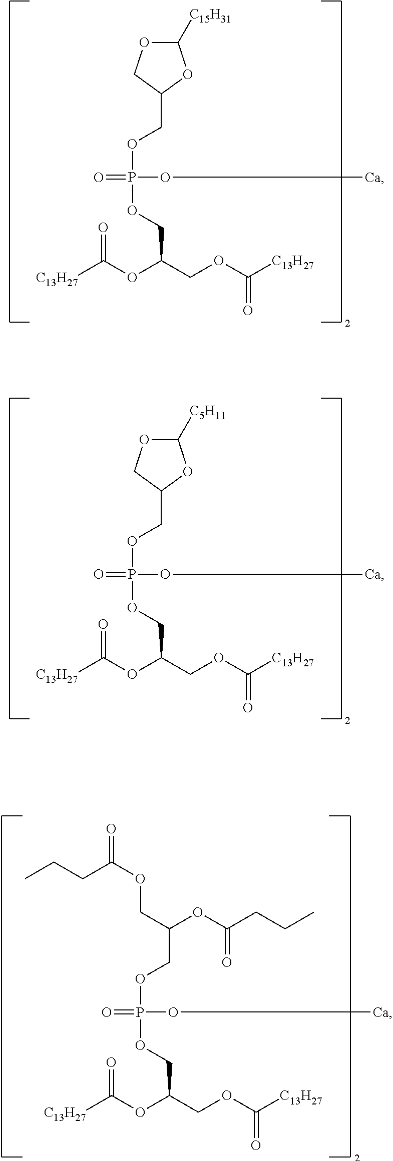

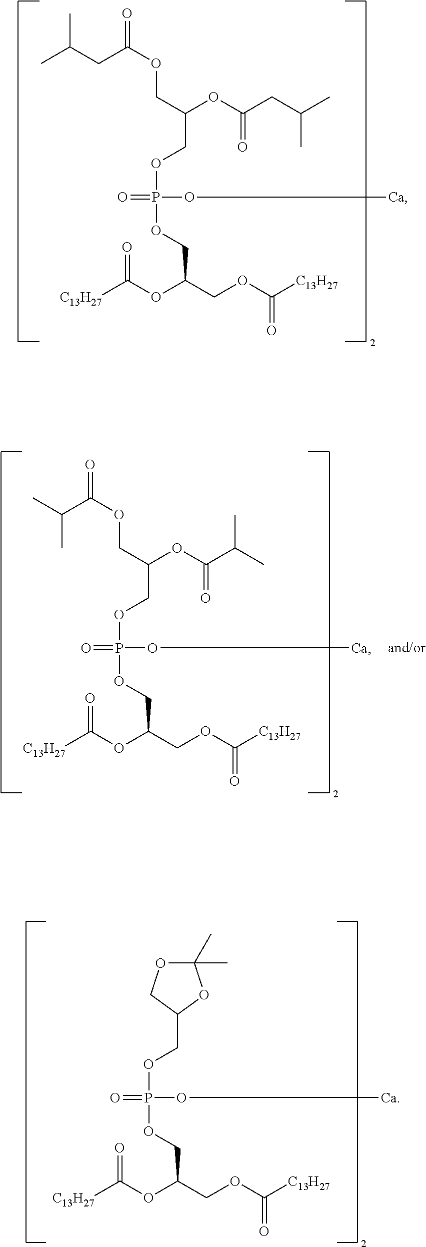

In another aspect, the compound is a single entity, a solvate, a hydrate, a crystal, an amorphous solid, a liquid or an oil. In another aspect, the compound is administered in at least once, once per day, twice per day, three times per day. In another aspect, the compound is administered at 0.1, 1, 2, 3, 4, 5, 6, 7, 89, 10, 15, 20, 25, 30, 40, 50, 60, 75, 80, 90, 100, 125, 150, 175, 200, 255, 250, 300, 400, or 500 mg/Kg. In another aspect, the composition is formulated into a pharmaceutical composition comprising one or more pharmaceutically acceptable excipients, buffers, or salts. In another aspect, the compound is formulated into a pharmaceutical composition adapted for oral, intravenous, nasal, pulmonary, alveolar, enteral, parenteral, or topical administration. In another aspect, the composition is formulated into an aerosol, a nebulizer, or an inhaler. In another aspect, the method further comprises one or more polymers, salts, or buffers. In another aspect, the method further comprises an additional therapeutic agent selected from the group consisting of corticosteroids, bronchodilators, anticholinergics, vasodilators, diuretics, anti-hypertensive agents, acetazolamide, antibiotics, antivirals, immunosuppressive drugs, and surfactants. In another aspect, the subject is a pediatric or adult human or a pediatric or adult animal. In another aspect, the compound is:

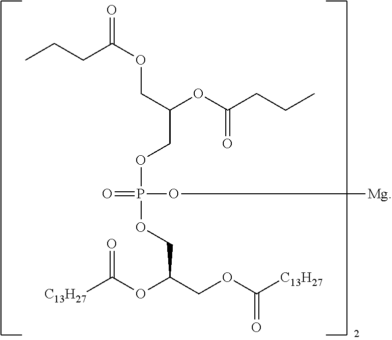

In another aspect, the method further comprises the step of identifying a subject in need of treatment for a pulmonary inflammation, distress or insufficiency prior to the treatment.

### BRIEF DESCRIPTION OF THE DRAWINGS

For a more complete understanding of the features and advantages of the present invention, reference is now made to the detailed description of the invention along with the accompanying figures and in which:

FIG. 1A shows body weight loss is significantly reduced at 48 hours and 72 hours after LPS treatment in animals treated with SPPCT-800, and FIG. 1B shows that the lung weight/body weight ratio is reduced.

FIGS. 2A to 2E show: (FIG. 2A) Blood oxygen levels, (FIG. 2B): Inspiration times, (FIG. 2C): Expiration times, (FIG. 2D): Breathing rates, (FIG. 2E): Pulmonary Congestion Indexes. Red dot=mice treated with SPPCT-800; black dot=mice treated with LPS and vehicle; open dot=untreated mice.

FIGS. 3A to 3N show: 3-Lung Injury Scores (FIG. 3A) Lung Injury Scores at 24-hours; (FIGS. 3B+C) Histology at 24-hours in sham mice; (FIGS. 3D+E) Histology at 24-hours in mice that received LPS+vehicle; FIGS. 3F+G) Histology at 24-hours in mice that received SPP4040 as a preventative; (FIG. 3H) Lung injury scores at 72 hours; (FIGS. 3I+J) Histology at 72-hours in sham mice; (FIGS. 3K+L) Histology at 72 hours in mice that received LPS+vehicle; (FIGS. 3M+N) Histology at 72 hours in mice that received SPPCT-800.

FIGS. 4A to 4C show: (FIG. 4A) Protein content in BALF at 24-hours with SPPCT-800 given one-half hour before LPS (FIG. 4B) with SPP4040 given three hours after LPS (FIG. 4C) Total cell count in BALF at 24 hours.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “in vivo” refers to being inside the body. The term “in vitro” used as used in the present application is to be understood as indicating an operation carried out in a non-living system.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes (i) inhibiting the disease in a subject that is experiencing or displaying the pathology or symptomatology of the diseased (i.e., arresting further development of the pathology and/or symptomatology); or (ii) ameliorating the disease in a subject that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, the terms “effective amount” or “therapeutically effective amount” described herein means the amount of the subject compound that will elicit the biological or medical response of a tissue, system, animal or human that is being sought by the researcher, veterinarian, medical doctor or other clinician.

As used herein, the terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

As used herein the term “intravenous administration” includes injection and other modes of intravenous administration.

As used herein, the term “pharmaceutically acceptable” as used herein to describe a carrier, diluent or excipient must be compatible with the other ingredients of the formulation and not deleterious to the recipient thereof.

A dosage unit for use of the lipid of Formula (I) of the present invention, may be a single compound or mixtures thereof with other compounds. The compound may be mixed together, form ionic or even covalent bonds. The lipids of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the lipids of the present invention to a patient in need of therapy that includes pulmonary disease including but not limited to bronchopulmonary dysplasia, asthma, chronic obstructive pulmonary disease, bronchitis, chronic or acute bronchoconstriction, acute respiratory distress syndrome, acute lung injury, cytokine storm, or bronchiectasis. The lipids may also be administered as any one of known salt forms.

The lipid of Formula (I) is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the lipid may be formulated to provide, e.g., maximum and/or consistent dosing for the particular form for oral, rectal, topical, intravenous injection or parenteral administration. While the lipid may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

For example, the lipid may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with a non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The lipid may be administered in the form of a liposome, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The lipid of Formula (I) may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or poly ethyleneoxide-polylysine substituted with palmitoyl residues, mixtures thereof, and the like. Furthermore, the lipid may be coupled one or more biodegradable polymers to achieve controlled release of the lipid, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the lipid of Formula (I) and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagous, tachea, lungs and alveoli, the lipid may also be delivered as an intranasal form via use of a suitable intranasal vehicle. For dermal and transdermal delivery, the lipid may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of lipid may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the

treatment of cancer, which comprise one or more containers containing a pharmaceutical composition comprising a therapeutically effective amount of lipid. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorings and coloring agents. Parenteral and intravenous forms may also include minerals and other materials to make them compatible with the type of injection or delivery system chosen.

To investigate the role of the lipids of the present invention, such as SPPCT-800, as a treatment for ARDS, the inventors used a murine LPS model to measure histologic changes, lung and body weights, oxygen blood saturations and other pulmonary function parameters, BALF protein and cell counts, and pro-inflammatory cytokine levels in the plasma and BALF.

C57B16/N mice were obtained from Charles River Laboratories (Montreal, Quebec). Male mice, 20-25 g of body weight, were used throughout the studies. Animals were housed in specific pathogen□free conditions, and all experiments were approved by Institutional Animal Care and Use Committee (IPST_SL20200402-1). Animal studies are reported in compliance with AAALAC guidelines.

All delivered mice were kept for one week as an acclimatization period prior to performing any experiments. Animals were housed in a maximum of two mice per cage under a 12□h light/12□h dark cycle at a temperature of ˜20-22° C. and 40-60% humidity. Food and water were available ad libitum. Each cage of mice was blindly assigned for different treatments or maintained as a control group according to the experimental design. The individual who carried out the experiments was not blinded as to treatment, but data analysis and experiments were otherwise blinded to avoid any bias.

In the ARDS group, mice received Escherichia coli O111:B4 lipopolysaccharide (50 μg in 0.05 mL saline, i.t.), while, in the control group, the animals received an instillation of saline (0.05 mL, i.t.). For intratracheal instillation, mice were slightly anesthetized with isoflurane.

The lipid SPPCT-800 was dissolved in water (low dose—2 mg/ml; high dose—20 mg/ml). For the 24-hour study, two treatment approaches were used. Mice received a single dose of SPPCT-800 (200 mg/kg, per gavage) as prevention (30 minutes before the LPS instillation), or as a single dose of either 20 mg/kg or 200 mg/kg as therapy (3-hour after the LPS instillation). For the 72-hour study, mice received a total of 8 treatments of SPPCT-800 (200 mg/kg) starting at 3-hour after the LPS instillation. Disease progression in mice was assessed by the evaluation of pulmonary functions, blood oxygen saturation, and body weight change. On the terminal day of each study (24 hours or 72 hours), measurement of cytokine levels in the plasma and BALF, and histopathology evaluations were done.

SPPCT-800, with chemical formula [C₄₂H₇₈O₁₂P]₂Mg is:

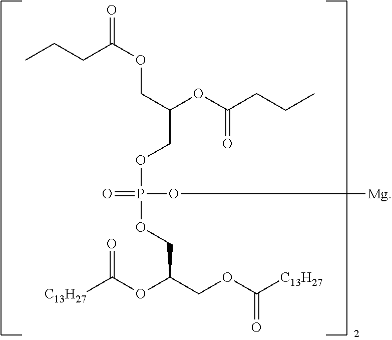

Other compounds for use with the present invention include the following compounds, along with phospholipids and phosphatidylglycerols in general, such as, 1,2-dimyristoyl-sn-glycero-3-phosphocholine (DMPC), 1,2-Dimyristoyl-sn-glycero-3-phosphoglycerol (DMPG), or DMPC/DMPG liposomes. In one aspect, the lysophosphatidylglycerol includes at least one of a lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine, and one or more of:

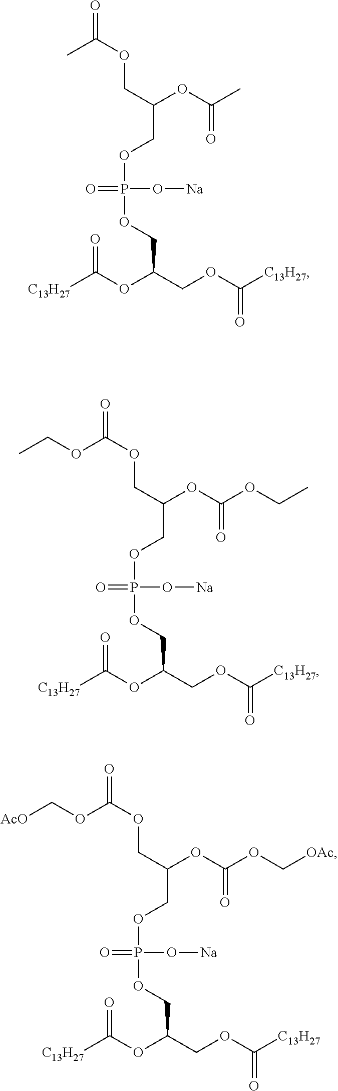

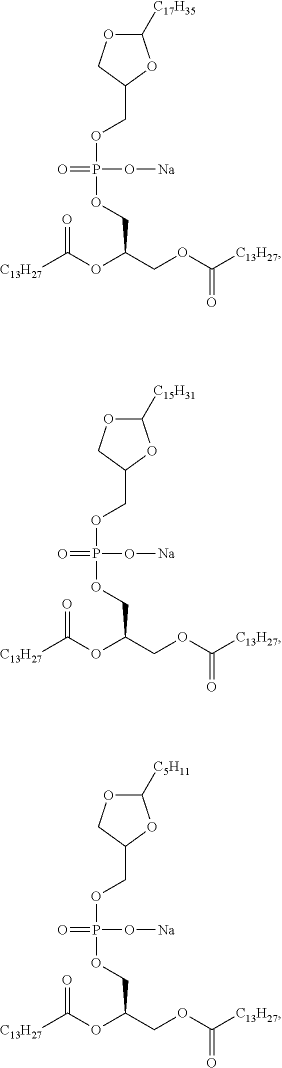

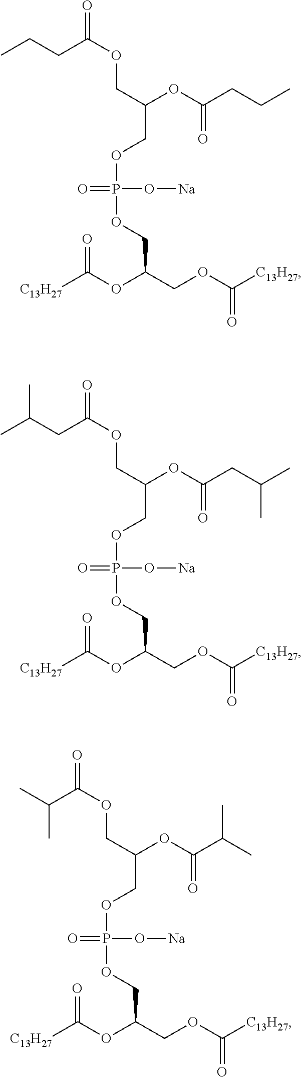

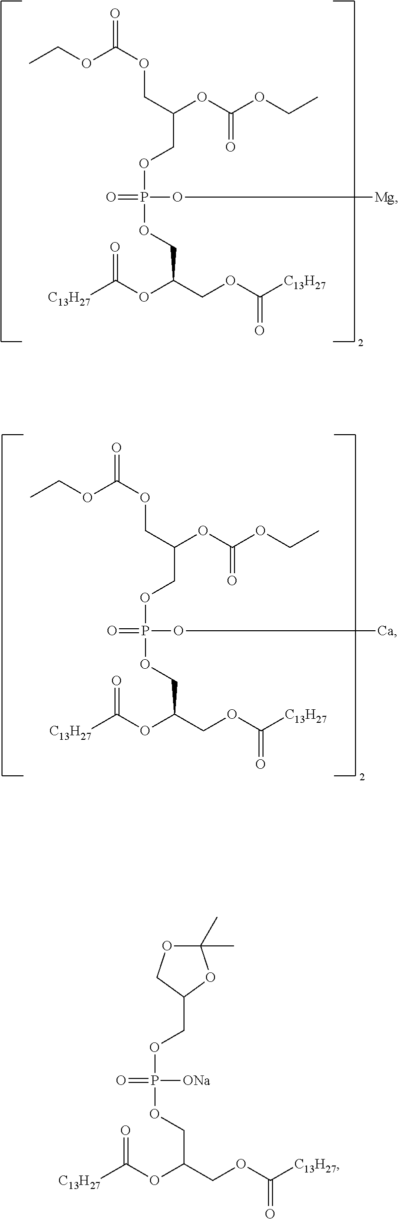

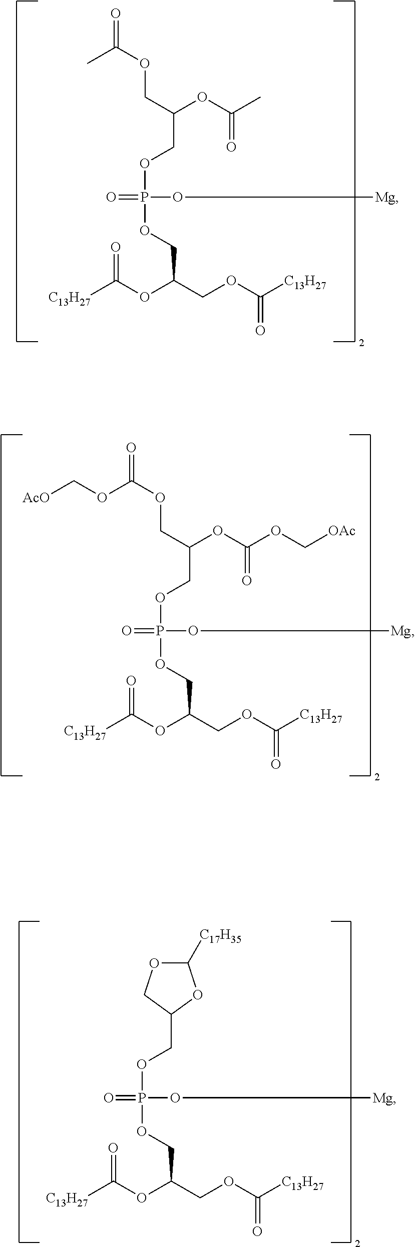

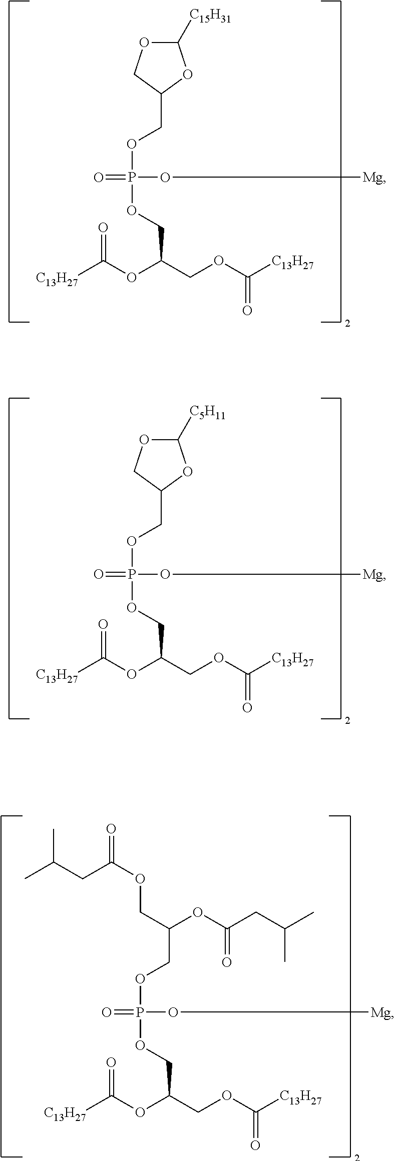

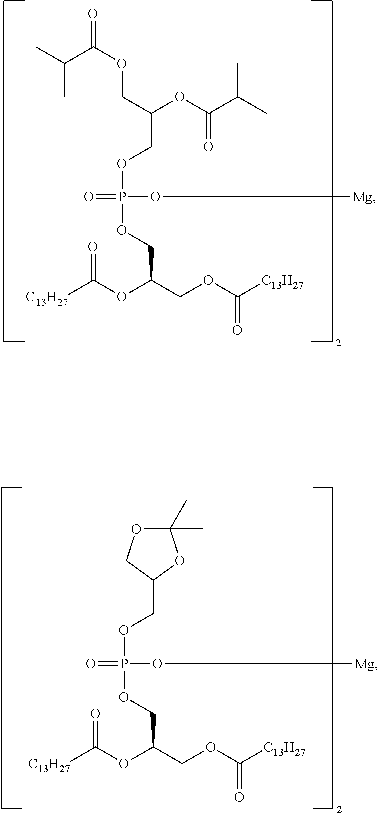

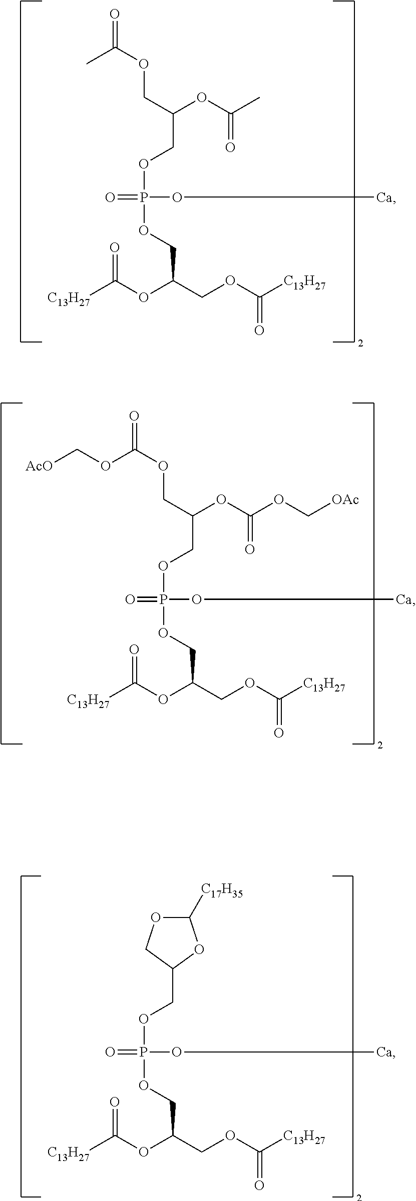

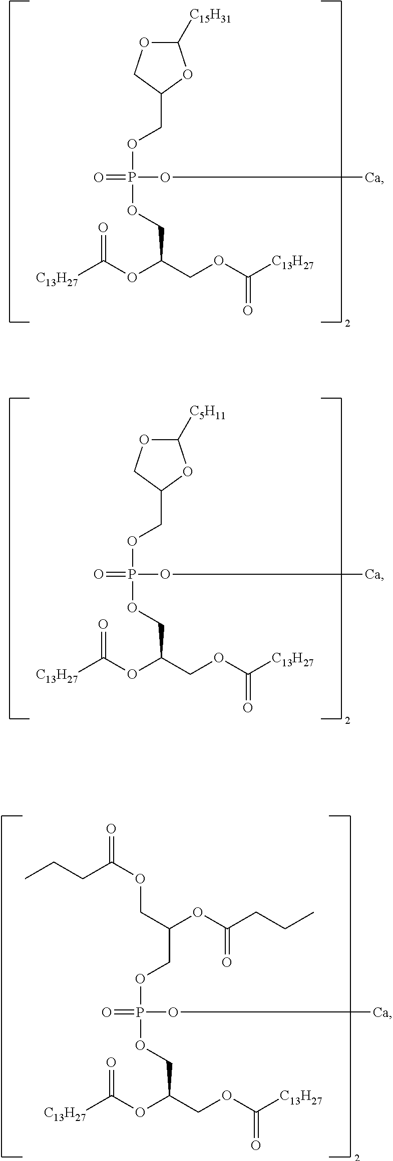

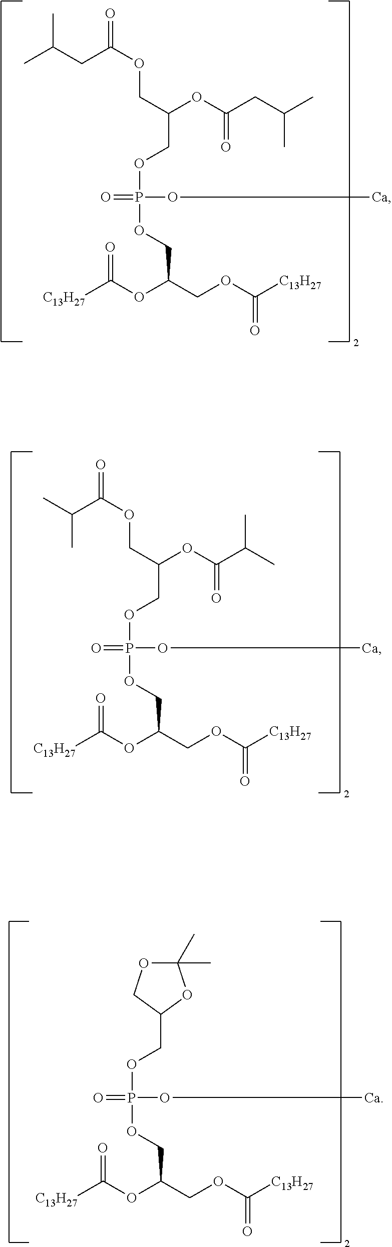

Respiratory Functions. All mice were introduced to the plethysmograph chamber environment. After the acclimatization period, the functional respiratory parameters were assessed by the whole-body plethysmograph (VivoFlow, SCIREQ, Montreal, Canada) at 0 hour (baseline), at 24-hour post-LPS instillation, and at 48 and 72-hour post-LPS instillation. Each measurement was performed with the mouse placed alone in an unrestrained whole-body plethysmography (WBP) chamber to measure respiratory functions. The WBP trace allowed us to determine specific information regarding the breathing pattern, and to derive important information associated with the development of inflammation. The functional respiratory parameters analyzed included respiratory rate, PenH (pulmonary congestion index), and inspiratory/expiratory time measurements. PenH, was used as an index of edema, inflammation and congestion (broncho-restriction).²⁴

At 0 hour (baseline), 24-hour post-LPS instillation, and 48 and 72-hour post LPS instillation, arterial blood oxygen saturation (SpO₂) was recorded on conscious mice. SpO₂ was read off of a pulse oximeter (STARR Life Sciences MouseOx Plus system, Oakmont, PA) with a mouse collar probe installed at the carotid level. The saturation values were measured in percentages (%).

Differential Cell Counts in BALF. The left lung was clamped while 0.9 mL of cold PBS 1×, Protease Inhibitor 1× (SigmaFast®) solution (3×300 μL) was injected so bronchoalveolar lavage fluid (BALF) from the right lobe of the lungs could be collected. The protein assay was performed according to the manufacturer's instruction BCA Protein Assay (Pierce™-#23227). Briefly, a dilution factor of 5 was used (1 part of BALF: 4 part of PBS 1×). 10 μL, of the diluted sample was added into the microplate wells. 200 μL of the working reagent was added into each well. The plate was covered, incubated at 37° C. for 30 minutes. After a cool-down period, the absorbance at 562 nm was rapidly measured using a monochromatic spectrophotometer (SpectraMAX ® plus—Molecular Devices). The total BALF protein content was reported by multiplying the protein concentration by the dilution factor, and then multiplying by the total of BALF volume collected.

Multiplex analysis of mediators. The inventors quantified 31 different mediators in the BALF and plasma by using a Discovery Assay® (Mouse Cytokine and Chemokine Array 31-Plex (MD31), Eve Technologies Corp, Calgary, AB, Canada). The multiplex assay was performed at Eve Technologies using the Bio-Plex™ 200 system (Bio-Rad Laboratories, Inc., Hercules, CA, USA) and a Milliplex Mouse Cytokine/Chemokine kit (Millipore, St. Charles, MO, USA) according to Eve Tech protocol. The 31-plex consisted of Eotaxin, G-CSF, GM-CSF, IFN-γ, IL-1α, IL-1β, IL-2, IL-3, IL-4, IL-5, IL-6, IL-7, IL-9, IL-10, IL-12 (p40), IL-12 (p70), IL-13, IL-15, IL-17, IP-10, KC, LIF, MCP-1, M-CSF, MIG, MIP-1α, MIP-1β, MIP-2,RANTES, TNFα, and VEGF. The assay sensitivities of these markers range from 0.1 pg/mL to 33.3 pg/mL. Individual analytes' values and other assay details are available on Eve Technologies' website and in the Milliplex protocol.

Histopathological Evaluation. The pulmonary airway was flushed with 0.9% NaCl, and the left lobe was inflated using a 10 mL syringe filled with a fixative (10% NBF) with an attached blunt tip needle (23G). The lung was gently inflated at a 20 cm H₂O pressure with fixative (10% NBF) until the lobe was fully, uniformly, and consistently expanded (not allowing fixative to ooze through the lung surface). This provided optimal airway expansion without causing tissue disruption. The left lobe was kept in fixative for 48 hours, and the 10% NBF was replaced by PBS and stored at 4° C. The left lung was embedded into paraffin blocks, which were sliced into 2 longitudinal slices of 5 μm thickness, and each slice were spaced by 50 μm in the middle part of the lung. After embedding and mounting of the tissues, the two slices were stained with Hematoxylin and Eosin (H&E). A blinded histologist evaluated the general morphology of alveolar septa, lung structure, and inflammation according to the general scoring described and adopted by Matute-Bello, et al.^{25, 26}

Statistical Analysis. Results are expressed as means±SEM. Comparisons were made on normally distributed data using ANOVA, followed by a Fisher post hoc test to assess the difference between LPS+vehicle group with GraphPad Prism Software version 8.0 (San Diego, CA, USA). Differences were considered statistically significant when P values were less than 0.05. (* Shown difference versus Sham animals and # shown difference versus LPS+Vehicle group. * Means P<0.05, ** P<0.01, *** P<0.001 while # means P<0.05, ##P<0.01 and ###P<0.001).

SPPCT-800 was effective against ARDS in all three studies. The best results were found in the 72-hour study. This is likely due to the fact that mice in the 72-hour study received a cumulative dose over the three days of 1600 mg/kg, which is eight times the single “high dose” (200 mg/kg) used in the 24-hour studies; although it could also be that the positive effects of SPPCT-800 are not fully seen at 24 hours.

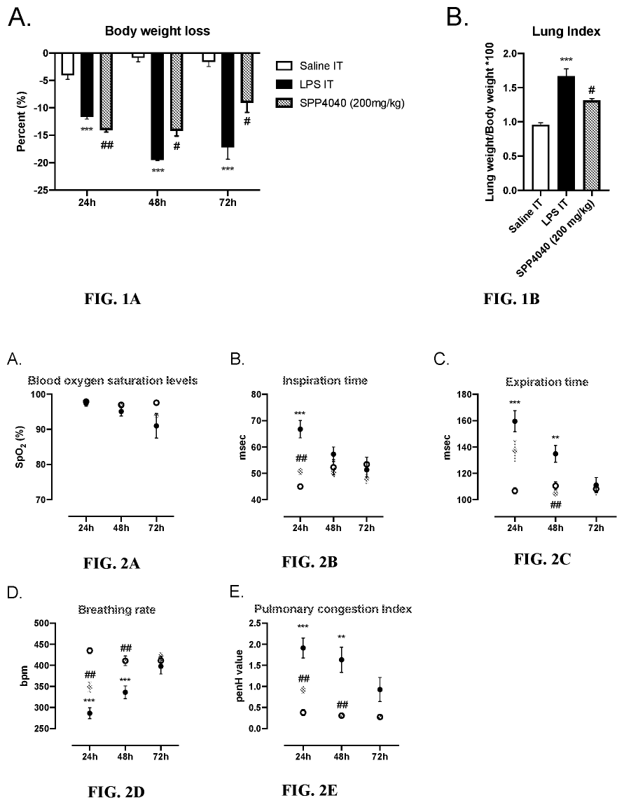

Physiologic and Respiratory Parameters. An important measure of a treatment's efficacy is the degree it reduces the weight loss in the animals with ARDS. At 24 hours, both the LPS mice and the LPS mice treated with SPPCT-800 showed a major loss of body weight compared to sham mice, with no protective effect from SPPCT-800 at that time (14% weight loss with SPPCT-800 compared to 12% with LPS alone). The weight loss at 48 hours stabilized at 14% in the SPPCT-800 group, while it reached 20% in the untreated LPS group (P<0.05). Importantly, at 72 hours, body weight loss decreased to 8% with SPPCT-800, compared to a loss of 18% in the untreated LPS group (P<0.05) (FIG. 1A). SPPCT-800 also tended to reduce the lung weight for animals sacrificed at 72 hours (P=0.1100). Thus, it significantly decreased the lung index (lung weight/body weight×100; P<0.05), and decreased the wet lung weight/dry lung ratio (FIG. 1B). Another important measure in ARDS patients is the maintenance of sufficient levels of oxygen in the arterial blood. In this 72-hour study, SpO₂ levels were maintained in the SPPCT-800 group compared to the untreated LPS group, where SpO₂ levels fell, though this result did not reach statistical significance. Improvement after SPPCT-800 treatment was also seen on pulmonary function tests at all measurement times. Significant reductions in inspiratory time and the pulmonary congestion index (PenH) at 24 hours. (P<0.001), as well as significant reductions in expiratory time and PenH, were seen at 48 hours (P<0.001) (FIG. 2).

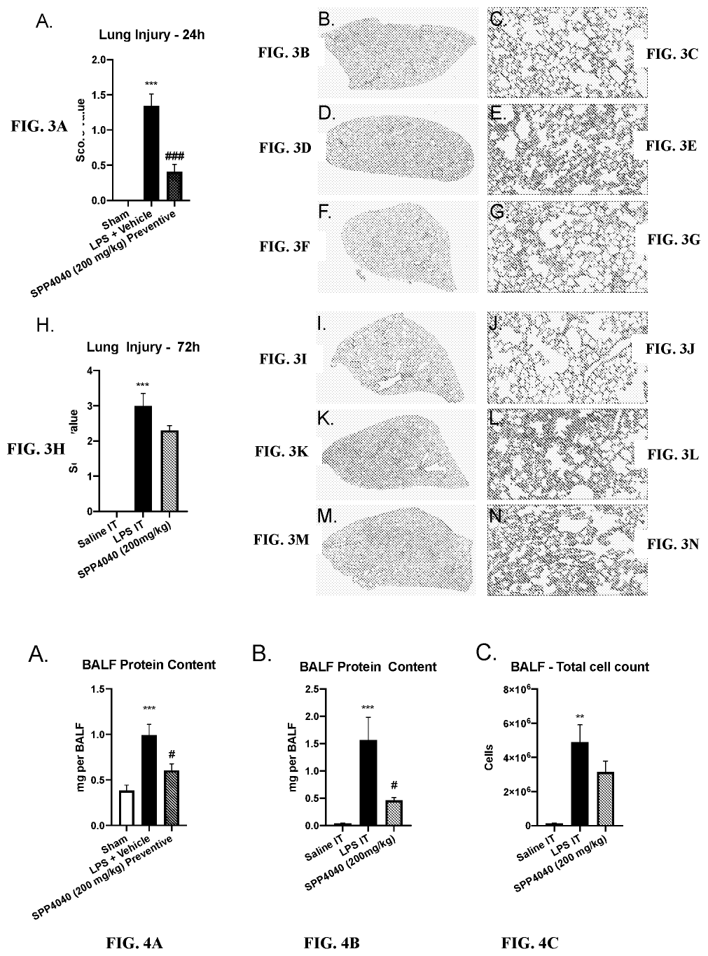

Lung Inflammation. Histological analysis, done as early as 24 hours showed SPPCT-800 could reduce the injury to the lung induced by LPS. SPPCT-800 given 30 minutes prior to LPS as a preventive significantly reduced the lung injury score value. The lung injury score at 24 hours was 1.4 when mice were given LPS alone, but was only 0.3 when SPPCT-800 was given as a preventative (P<0.01) (FIG. 3A). At 72 hours, the lung injury score was also reduced. Mice in the multi-dose SPPCT-800 treatment group had a reduced score of 2.2 compared to a score of 3.0 in the mice not given SPPCT-800 (FIG. 3H). Histologic changes are also shown in FIG. 3. A single dose of SPPCT-800 given as a preventive also gave evidence that it could reduce inflammation as early as 24 hours after LPS injection, with a significant reduction in BALF protein content (P<0.05). At 72 hours, in mice receiving multidose SPPCT-800 treatment, there were major reductions in BALF protein levels, as well as reductions in BALF total cell count (P=0.1714) and BALF neutrophil counts (P=0.1493), which did not reach statistical significance (FIG. 4).

Pro-inflammatory Cytokines. Dramatic reductions in the pro-inflammatory cytokines tested were seen after treatment with SPPCT-800, at 24 hours and at 72 hours, in both the plasma and the BALF (see Table 1 and Table 2). Major reductions in TNF-α, INF-γ, G-CSF, GM-CSF, interleukins, VEGF, MCP-1, KC and MIP-1α were seen in all three groups of mice in the single-dose 24-hour study (200 mg/mL preventative, 20 mg/mL therapeutic, 200 mg/mL therapeutic), and in the repeat dose 72-hour study. For example, the very important cytokine, TNF-α, was markedly reduced at 24 hours in the plasma by SPPCT-800 in all three of the above single dose groups (P<0.001), and in the BALF (P=0.0959). Similarly, in the 72-hour study, TNF-α levels were markedly reduced in the plasma (P<0.05) and the BALF (p=0.0613).

TABLE 1
Cytokine levels in plasma measured at 24-hours after sham injection, after LPS-vehicle
injection, after LPS preceded by SPP4040 by 30 minutes, and after LPS followed 3 hours
later by SPP4040. Cytokine levels in plasma measured at 72-hours after LPS+ vehicle
injection, and after LPS injection followed by eight SPP4040 injections over 72 hours.

| | | 24-hours | | | 72-hours | |
|---|---|---|---|---|---|---|
| | | LPS- | SPP-4040 | SPP-4040 | LPS- | SPP-4040 |
| | Sham | vehicle | Pre | Thera | vehicle | Thera |
| Eotaxin | 456 ± 125 | 428 ± 28 | 719 ± 138 | 855 ± 228 | 341 ± 38 | 464 ± 252 |
| G-CSF | 198 ± 29 | 25015 ± 889 | 21035 ± 2241 | 22915 ± 410 | 5720 ± 2510 | 1116 ± 502 |
| GM-CSF | 6 ± 1 | 46 ± 3 | 12 ± 2 | 11 ± 1 | 49 ± 9 | 6 ± 2 |
| IFN gamma | 1 ± 1 | 9 ± 2 | 1 ± 1 | 1 ± 1 | 29 ± 13 | 6 ± 2 |
| IL1-alpha | 101 ± 33 | 303 ± 26 | 3196 ± 3107 | 159 ± 39 | 161 ± 31 | 63 ± 27 |
| IL1-beta | 1.5 ± 0.1 | 12.7 ± 6.2 | 3.8 ± 0.7 | 3.6 ± 0.6 | 5.3 ± 1.5 | 6.8 ± 3.8 |
| IL2 | 6 ± 3 | 36 ± 7 | 11 ± 3 | 16 ± 1 | 52 ± 11 | 34 ± 10 |
| IL3 | 0.4 ± 0.1 | 2.8 ± 0.8 | 0.9 ± 0.1 | 0.5 ± 0.7 | 1.3 ± 0.6 | 0.1 ± 0.1 |
| IL4 | 0.3 ± 0.2 | 1.5 ± 0.7 | 0.2 ± 0.1 | 0.4 ± 0.2 | 5.9 ± 4.4 | 0.6 ± 0.4 |
| IL5 | 4 ± 1 | 32 ± 5 | 8 ± 2 | 11 ± 2 | 22 ± 5 | 2 ± 1 |
| IL6 | 2 ± 1 | 641 ± 228 | 176 ± 29 | 155 ± 30 | 539 ± 213 | 76 ± 31 |
| IL7 | 3 ± 1 | 34 ± 25 | 8 ± 2 | 4 ± 1 | 7 ± 1 | 3 ± 1 |
| IL9 | 8 ± 2 | 52 ± 8 | 2 ± 1 | 6 ± 2 | 46 ± 18 | 3 ± 1 |
| IL10 | 4 ± 1 | 128 ± 23 | 57 ± 8 | 90 ± 32 | 40 ± 17 | 5 ± 1 |
| IL12 (p40) | 7 ± 2 | 21 ± 3 | 12 ± 1 | 12 ± 2 | 12 ± 1 | 8 ± 1 |
| IL12 (p70) | 8 ± 2 | 37 ± 15 | 17 ± 7 | 40 ± 19 | 98 ± 71 | 11 ± 5 |
| IL13 | 16 ± 1 | 72 ± 6 | 22 ± 3 | 22 ± 2 | 72 ± 16 | 23 ± 3 |
| IL15 | 19 ± 14 | 408 ± 243 | 63 ± 15 | 49 ± 16 | 124 ± 17 | 40 ± 22 |
| IL17 | 1.0 ± 0.2 | 14.2 ± 10.4 | 2.6 ± 0.3 | 2.3 ± 0.6 | 11 ± 4 | 4 ± 3 |
| IP-10 | 63 ± 6 | 504 ± 52 | 790 ± 206 | 605 ± 102 | 300 ± 66 | 150 ± 34 |
| KC | 51 ± 21 | 3933 ± 1362 | 1271 ± 224 | 764 ± 274 | 1049 ± 358 | 502 ± 150 |
| LIF | 0.6 ± 0.1 | 10.5 ± 8.2 | 1.3 ± 0.2 | 1.5 ± 0.2 | 2.2 ± 0.7 | 0.5 ± 0.1 |
| LIX | 71 ± 23 | 3067 ± 874 | 470 ± 145 | 547 ± 203 | 2117 ± 529 | 574 ± 445 |
| MCP-1 | 11 ± 1 | 157 ± 22 | 86 ± 20 | 157 ± 62 | 55 ± 7 | 34 ± 6 |
| M-CSF | 6 ± 1 | 42 ± 4 | 12 ± 1 | 16 ± 4 | 24 ± 1 | 5 ± 2 |
| MIG | 557 ± 201 | 1261 ± 121 | 2486 ± 203 | 2593 ± 317 | 353 ± 122 | 447 ± 137 |
| MIP-1alpha | 38 ± 5 | 197 ± 28 | 88 ± 4 | 92 ± 7 | 210 ± 14 | 133 ± 11 |
| MIP-1beta | 44 ± 7 | 121 ± 11 | 109 ± 8 | 120 ± 29 | 102 ± 16 | 55 ± 13 |
| MIP-2 | 91 ± 7 | 86 ± 13 | 138 ± 7 | 128 ± 5 | 61 ± 8 | 56 ± 17 |
| RANTES | 15 ± 4 | 360 ± 45 | 200 ± 28 | 270 ± 70 | 64 ± 13 | 19 ± 2 |
| TNF-alpha | 5 ± 1 | 36 ± 2 | 14 ± 1 | 14 ± 1 | 28 ± 7 | 7 ± 2 |
| VEGF | 0.4 ± 0.1 | 1.3 ± 0.3 | 0.5 ± 0.1 | 0.6 ± 0.1 | 28.0 ± 7.0 | 7.4 ± 2.4 |

TABLE 2
Cytokine levels in BALF measured at 24-hours after sham injection, after LPS-vehicle
injection, after LPS preceded by SPP4040 by 30 minutes, and after LPS followed 3 hours
later by SPP4040. Cytokine levels in BALF measured at 72-hours after LPS+ vehicle
injection, and after LPS injection followed by eight SPP4040 injections over 72 hours.

| | | 24-hours | | | 72-hours | |
|---|---|---|---|---|---|---|
| | | LPS- | SPP-4040 | SPP-4040 | LPS- | SPP-4040 |
| | Sham | vehicle | Pre | Thera | vehicle | Thera |
| Eotaxin | 3 ± 1 | 15 ± 3 | 10 ± 1 | 14 ± 3 | 24 ± 8 | 6 ± 2 |
| G-CSF | 39 ± 8 | 8034 ± 699 | 6468 ± 381 | 7060 ± 853 | 3648 ± 1039 | 1233 ± 572 |
| GM-CSF | 16 ± 1 | 81 ± 6 | 26 ± 5 | 46 ± 9 | 48 ± 5 | 9 ± 1 |
| IFN gamma | 7 ± 1 | 10±1 | 2 ± 1 | 3 ± 1 | 34 ± 14 | 12 ± 1 |
| IL1-alpha | 38 ± 3 | 220 ± 12 | 183 ± 25 | 331 ± 67 | 494 ± 153 | 576 ± 169 |
| IL1-beta | 1 ± 1 | 190 ± 82 | 69 ± 9 | 103 ± 18 | 174 ± 42 | 89 ± 24 |
| IL2 | 5.5 ± 0.3 | 9.6 ± 0.5 | 1.5 ± 0.2 | 1.5 ± 0.3 | 9.2 ± 2.1 | 1.7 ± 0.4 |
| IL3 | 1.4 ± 0.1 | 3.2 ± 0.3 | 0.4 ± 0.1 | 0.5 ± 0.1 | 3.5 ± 0.8 | 0.5 ± 0.2 |
| IL4 | 0.06 ± 0.03 | 0.36 ± 0.06 | 0.08 ± 0.01 | 0.10 ± 0.01 | 0.74 ± 0.16 | 0.24 ± 0.05 |
| IL5 | 29 ± 6 | 39 ± 6 | 10 ± 3 | 10 ± 2 | 12 ± 4 | 0.4 ± 0.1 |
| IL6 | 11 ± 3 | 942 ± 194 | 856 ± 188 | 850 ± 212 | 4664 ± 2591 | 276 ± 151 |
| IL7 | 2.8 ± 0.2 | 3.7 ± 0.2 | 1.7 ± 0.3 | 1.7 ± 0.2 | 3.6 ± 0.6 | 2.0 ± 0.5 |
| IL9 | 9.8 ± 1.1 | 14.5 ± 1.3 | 7.0 ± 0.3 | 8.8 ± 1.3 | 16.7 ± 3.3 | 8.5 ± 1.6 |
| IL10 | 1.2 ± 0.1 | 3.5 ± 0.4 | 1.4 ± 0.3 | 1.4 ± 0.3 | 9.0 ± 6.7 | 1.1 ± 0.5 |
| IL12 (p40) | 1.2 ± 0.2 | 3.8 ± 0.3 | 1.6 ± 0.3 | 2.0 ± 0.3 | 6.2 ± 2.0 | 6.0 ± 1.2 |
| IL12 (p70) | 9.5 ± 0.9 | 15.6 ± 1.8 | 7.4 ± 1.0 | 6.0 ± 1.0 | 26.5 ± 6.8 | 15.7 ± 3.9 |
| IL13 | 0.5 ± 0.1 | 4.4 ± 0.5 | 0.7 ± 0.1 | 1.1 ± 0.2 | 7.4 ± 2.1 | 1.1 ± 0.2 |
| IL15 | 9.7 ± 1.4 | 16.8 ± 1.5 | 5.1 ± 0.8 | 3.1 ± 0.7 | 12.8 ± 3.8 | 4.8 ± 2.6 |
| IL17 | 0.2 ± 0.1 | 10.6 ± 2.7 | 8.3 ± 3.2 | 13.6 ± 5.6 | 102.9 ± 75.6 | 53.2 ± 26.9 |
| IP-10 | 9 ± 1 | 115 ± 13 | 125 ± 10 | 163 ± 33 | 1061 ± 339 | 1583 ± 385 |
| KC | 20 ± 2 | 932 ± 105 | 622 ± 89 | 1017 ± 140 | 714 ± 143 | 216 ± 15 |
| LIF | 2 ± 1 | 39 ± 8 | 38 ± 8 | 39 ± 12 | 103 ± 55 | 11 ± 7 |
| LIX | 3 ± 1 | 74 ± 5 | 94 ± 6 | 67 ± 20 | 91 ± 24 | 25 ± 18 |
| MCP-1 | 10 ± 1 | 372 ± 75 | 462 ± 103 | 494 ± 103 | 624 ± 461 | 84 ± 34 |
| M-CSF | 2 ± 1 | 30 ± 5 | 21 ± 3 | 27 ± 6 | 21 ± 5 | 11 ± 1 |
| MIG | 3.4 ± 1.0 | 16.7 ± 4.0 | 6.8 ± 1.5 | 6.6 ± 2.0 | 346.8 ± 212.7 | 749.7 ± 186.7 |
| MIP-1alpha | 18 ± 3 | 447 ± 58 | 323 ± 52 | 564 ± 132 | 1082 ± 274 | 1676 ± 554 |
| MIP-1beta | 19 ± 1 | 536 ± 105 | 801 ± 208 | 1924 ± 812 | 1138 ± 513 | 1561 ± 440 |
| MIP-2 | 33 ± 3 | 1410 ± 950 | 771 ± 107 | 1054 ± 106 | 3647 ± 2324 | 2649 ± 898 |
| RANTES | 2 ± 1 | 30 ± 3 | 45 ± 6 | 46 ± 6 | 38.17 ± 12 | 57 ± 20 |
| TNF-alpha | 1 ± 1 | 185 ± 31 | 91 ± 32 | 153 ± 32 | 173 ± 51 | 62 ± 17 |
| VEGF | 13 ± 2 | 41 ± 3 | 33 ± 2 | 34 ± 2 | 29 ± 7 | 11 ± 3 |

SPPCT-800 profoundly depressed levels of another key cytokine, IFN-γ, in all of the mouse groups, in all three single-dose groups at 24 hours in the plasma (P<0.01 in all three groups), and in the BALF (P<0.001). Large reductions in IFN-γ levels were also seen at 72 hours, in both the plasma and the BALF.

GM-CSF, which causes the secretion of neutrophils, monocytes and other cells, and is, thus, thought to play a major role in causing lung damage in ARDS patients, was markedly reduced in all mice treated with SP4040. In all three mouse groups at 24 hours (200 mg/kg preventive, 20 mg/kg therapeutic and 200 mg/kg therapeutic), there were highly significant depressions of GM-CSF by SPPCT-800 (P<0.001) in both the plasma and the BALF. Likewise, at 72 hours, significant decreases of GM-CSF were noted in both the plasma (P<0.01) and the BALF (P<0.001).

Of the 15 interleukins tested, major reductions with SPPCT-800 were consistently noted in 14. At 24 hours, IL-1β, IL-2 (P<0.05), IL-3 (P=0.0859 in 200 mg therapeutic group), IL-4, IL-5 (P<0.05), IL-6 (P=0.0624), IL-7, IL-9 (P<0.001), IL-10 (P=0.0767 in the preventive group), IL-12 (p 40) (P=0.1101 in the preventive group), IL-12 (p70), IL-13 (P<0.001), IL-15 and IL-17 were markedly reduced. SPPCT-800 did not reduce levels of plasma IL-1α in any of the mouse groups at 24 hours. With the exception of IL-6, similar major reductions in these interleukins at 24 hours were seen in the BALF, with major reductions in IL-1β, IL-2 (P<0.001), IL-3 (P<0.001), IL-4 (P<0.05), IL-5 (P<0.01), IL-7 (P<0.001), IL-9 (P<0.01), IL-10 (P<0.01), IL-12 (p40) (P<0.001), IL-12 (p70) (P<0.01), IL-13 (P<0.001) and IL-15 (P<0.001). Significant results, at 72 hours in plasma and in the BALF, were also found. SPPCT-800 also suppressed G-CSF, VEGF, KC, LIF (leukemia inhibitory factor), LIX (LPS-induced CXC chemokine 5), and M-CSF (macrophage colony stimulating factor), in the plasma and the BALF. MCP-1 was inhibited by SPPCT-800 in the plasma at 24 hours, and in both the plasma and BALF at 72 hours. Suppressive effects against MIP-1α and MIP 1-β (macrophage inflammatory protein 1-β) were found at 24 hours in both the plasma and BALF and also at 72 hours in the plasma. RANTES (Regulated on Activation, Normal T cell Expressed and Secreted; CCL 5) was suppressed in the plasma at both 24 hours and 72 hours, but not in the BALF. Results were mixed, with marked suppression by SPPCT-800 in the plasma or the BALF, but not in both, against MIG (monokine induced by human interferon), IP10 and Eotaxin, an eosinophil chemotactic protein.

The present invention demonstrates the protective and therapeutic effects of the lipids of the present invention, such as SPPCT-800, against ARDS were shown by multiple measures. The mice treated with this agent demonstrated decreased lung damage and definite clinical improvement, with decreased weight loss, improved pulmonary function tests and oxygen saturation levels, and better lung injury scores. Protein content and neutrophil cell counts in the BALF were reduced. Correspondingly, inflammation was profoundly suppressed in the animals treated with SPP4040. The key cytokines, TNF-α, crucial in the etiology of numerous diseases, and interferon γ, central for its capacity to be a suppressant of indoleamine 2,3-dioxygenase²⁷, as well as almost all interleukins, were profoundly suppressed. Levels of VEGF, thought to play a major role in the massive lung damage and lung edema seen with ARDS, were also markedly reduced (28). G-CSF and GM-CSF, which are used to stimulate neutrophils in patients receiving chemotherapy, but can induce an ARDS-like syndrome²⁹⁻³², were also dramatically suppressed.

Increased inflammation is a crucial part of the development of many diseases, including cancer^{33, 34}, Parkinson's disease²⁵ and coronary heart disease^{36, 37}. Much as the cerebral damage after many neurologic injuries is due to a massive accumulation of cytokines within the brain³⁸, ARDS can be thought of similarly, as a massive accumulation of cytokines within the lungs. Sepsis, itself, can be interpreted as a whole-body inflammatory process brought on by systemic infections. Thus, multiple studies have been done in ARDS patients of medications that suppress cytokines. Clinical trials with anti-inflammatory agents have given equivocal results, and many of these medications subject patients to additional toxicities^{2, 39-41}. Corticosteroids have been the most commonly used treatment in patients with ARDS, although many new treatments, including antivirals, are being introduced to treat the subset of patients with ARDS secondary to COVID-19. Again, treatment with corticosteroids is controversial, has not been proven to increase survival, and exposes the patient to numerous additional risks, including severe hyperglycemia, hypokalemia, GI bleeding, severe hypertension, and fungal or bacterial infections⁴²⁻⁴⁴.

SPPCT-800, with chemical formula [C₄₂H₇₈O₁₂P]₂Mg, is a white crystalline powder which can be taken orally. It has detectable activity after a single dose of 1 mg/kg, and is non-toxic at doses of up to 800 mg/kg per day. This study demonstrates that, in the in vivo murine LPS ARDS model that a single dose of 200 mg/kg of SPPCT-800, given 30 minutes before LPS challenge, significantly reduces severe damage to the lung by suppressing inflammation. Further, in this model, therapeutic doses of SPPCT-800 were shown to be effective in treating

ARDS and reducing lung damage.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Thompson B T, Chambers R C, Liu K D. Acute respiratory distress syndrome. NEJM. 2017 Aug. 10; 377(6):562-72.
2. Dushianthan A, Grocott M P, Postle A D, Cusack R. Acute respiratory distress syndrome and acute lung injury. PMJ. 2011 Sep. 1; 87(1031):612-22.
3. Confalonieri M, Salton F, Fabiano F. Acute respiratory distress syndrome. ERR. 2017 Jun. 30; 26(144):160116.
4. Kirch C, Blot F, Fizazi K, Raynard B, Theodore C, Nitenberg G. Acute respiratory distress syndrome after chemotherapy for lung metastases from non-seminomatous germ-cell tumors. SCC. 2003 Sep. 1; 11(9):575-80.
5. Xu Z, Shi L, Wang Y, Zhang J, Huang L, Zhang C, Liu S, Zhao P, Liu H, Zhu L, Tai Y. Pathological findings of COVID-19 associated with acute respiratory distress syndrome. Lancet Respir Med. 2020 Apr. 1; 8(4):420-2.
6. Grasselli G, Tonetti T, Protti A, Langer T, Girardis M, Bellani G, Laffey J, Carrafiello G, Carsana L, Rizzuto C, Zanella A. Pathophysiology of COVID-19-associated acute respiratory distress syndrome: a multicentre prospective observational study. Lancet Respir Med. 2020 Dec. 1; 8(12):1201-8.
7. Spadaro S, Park M, Turrini C, Tunstall T, Thwaites R, Mauri T, Ragazzi R, Ruggeri P, Hansel T T, Caramori G, Volta C A. Biomarkers for acute respiratory distress syndrome and prospects for personalised medicine. J Inflamm. 2019 Dec. 1; 16(1):1.
8. Ware L B, Matthay M A. The acute respiratory distress syndrome. NEJM. 2000 May 4; 342(18):1334-49.

9. Juss J K, House D, Amour A, Begg M, Herre J, Storisteanu D M, Hoenderdos K, Bradley G, Lennon M, Summers C, Hessel E M. Acute respiratory distress syndrome neutrophils have a distinct phenotype and are resistant to phosphoinositide 3-kinase inhibition. Am J Respir Crit Care Med. 2016 Oct. 15; 194(8):961-73.
10. Matute-Bello G, Liles W C, RADELLA F, Steinberg K P, Ruzinski J T, Jonas M, Chi E Y, Hudson L D, Martin T R. Neutrophil apoptosis in the acute respiratory distress syndrome. Am J Respir Crit Care Med. 1997 Dec. 1; 156(6):1969-77.
11. Windsor A C, Mullen P G, Fowler A A, Sugerman H J. Role of the neutrophil in adult respiratory distress syndrome. BJS. 1993 January; 80(1):10-7.
12. Wang Y, Ju M, Chen C, Yang D, Hou D, Tang X, Zhu X, Zhang D, Wang L, Ji S, Jiang J. Neutrophil-to-lymphocyte ratio as a prognostic marker in acute respiratory distress syndrome patients: a retrospective study. J Thorac Dis. 2018 January; 10(1):273.
13. Ma A, Cheng J, Yang J, Dong M, Liao X, Kang Y. Neutrophil-to-lymphocyte ratio as a predictive biomarker for moderate-severe ARDS in severe COVID-19 patients. Crit Care. 2020 December; 24(1):1-4.
14. Williams A E, Chambers R C. The mercurial nature of neutrophils: still an enigma in ARDS?. Am J Physiol Lung Cell Mol. 2014 Feb. 1; 306(3):L217-30.
15. Scott B N, Kubes P. Death to the neutrophil! A resolution for acute respiratory distress syndrome?. Eur Respir J. 2018; 52:1801274.
16. Yang S C, Tsai Y F, Pan Y L, Hwang T L. Understanding the role of neutrophils in acute respiratory distress syndrome. Biomed J. 2020 Sep. 10.
17. Rebetz J, Semple J W, Kapur R. The pathogenic involvement of neutrophils in

acute respiratory distress syndrome and transfusion-related acute lung injury. Transfus Med Hemother. 2018; 45(5):290-8.
18. Chen K, Kolls J K. Innate Lymphoid Cells and Acute Respiratory Distress Syndrome. Am J Respir Crit Care Med. 2016 Feb. 15; 193(4):350-2.
19. Meduri G U, Kohler G, Headley S, Tolley E, Stentz F, Postlethwaite A. Inflammatory cytokines in the BAL of patients with ARDS: persistent elevation over time predicts poor outcome. Chest. 1995 Nov. 1; 108(5):1303-14.
20. Preira P, Forel J M, Robert P, Nègre P, Biarnes-Pelicot M, Xeridat F, Bongrand P, Papazian L, Theodoly O. The leukocyte-stiffening property of plasma in early acute respiratory distress syndrome (ARDS) revealed by a microfluidic single-cell study: the role of cytokines and protection with antibodies. Crit Care. 2015 Dec. 1; 20(1):8.
21. Wilson J G, Simpson L J, Ferreira A M, Rustagi A, Roque J, Asuni A, Ranganath T, Grant P M, Subramanian A, Rosenberg-Hasson Y, Maecker H T. Cytokine profile in plasma of severe COVID-19 does not differ from ARDS and sepsis. MedRxiv. 2020 Jan. 1.
22. Juskewitch J E, Knudsen B E, Platt J L, Nath K A, Knutson K L, Brunn G J, Grande J P. LPS-induced murine systemic inflammation is driven by parenchymal cell activation and exclusively predicted by early MCP-1 plasma levels. Am J Pathol. 2012 Jan. 1; 180(1):32-40.
23. Henderson W R, Chen L, Amato M B, Brochard L J. Fifty years of research in ARDS. Respiratory mechanics in acute respiratory distress syndrome. Am J Respir Crit Care Med. 2017 Oct. 1; 196(7):822-33.
24. Lomask M. Further exploration of the Penh parameter. Toxicol Pathol. 2006 Jun. 15; 57:13-20.
25. Matute-Bello G, Frevert C W, Martin T R. Animal models of acute lung injury. Am J Physiol Lung Cell Mol. 2008 September; 295(3):L379-99.
26. Aeffner F, Bolon B, Davis I C. Mouse models of acute respiratory distress syndrome: a review of analytical approaches, pathologic features, and common measurements. Toxicol Pathol. 2015 December; 43(8):1074-92.
27. Sordillo L A, Sordillo P P. Optical spectroscopy of tryptophan metabolites in neurodegenerative disease. In: Alfano R R, Shi L, eds. Neurophotonics and Biomedical Spectroscopy. Elsevier; 2019: 137-157.
28. Barratt S, Medford A R, Millar A B. Vascular endothelial growth factor in acute lung injury and acute respiratory distress syndrome. Respiration. 2014; 87(4):329-42.
29. Kudlak K, DeMuro J P, Hanna A F, Brem H. Acute lung injury following the use of granulocyte-macrophage colony-stimulating factor. Int J Crit Illn Inj Sci. 2013 October; 3(4):279.
30. Inokuchi R, Manabe H, Ohta F, Nakamura K, Nakajima S, Yahagi N. Granulocyte colony□stimulating factor□producing lung cancer and acute respiratory distress syndrome. Clin Respir J. 2015 April; 9(2):250-2.
31. Takatsuka H, Takemoto Y, Mori A, Okamoto T, Kanamaru A, Kakishita E. Common features in the onset of ARDS after administration of granulocyte colony-stimulating factor. Chest. 2002 May 1; 121(5):1716-20.
32. Rhee C K, Kang J Y, Kim Y H, Kim J W, Yoon H K, Kim S C, Kwon S S, Kim Y K, Kim K H, Moon H S, Park S H. Risk factors for acute respiratory distress syndrome during neutropenia recovery in patients with hematologic malignancies. Crit Care. 2009 Dec. 1; 13(6):R173.
33. Sordillo P P, Sordillo L A. Glioblastoma cell-induced immunosuppression causing chemoresistance. In: Massoud and Paulmurugan Eds, Glioblastoma Resistance to Chemotherapy: Molecular mechanisms and innovative reversal strategies,. Elsevier, Cambridge, MA (in press).
34. Greten F R, Grivennikov S I. Inflammation and cancer: triggers, mechanisms, and consequences. Immunity. 2019 Jul. 16; 51(1):27-41.
35. Ferrari C C, Tarelli R. Parkinson's disease and systemic inflammation. Parkinson's Dis. 2011 Feb. 22; 2011.
36. Sordillo P P, Sordillo D C, Helson L. The prolonged QT Interval: role of pro-inflammatory cytokines, reactive oxygen species and the ceramide and sphingosine-1 phosphate pathways. In Vivo. 2015 Nov. 1; 29(6):619-36.
37. Stanciu A E. Cytokines in heart failure. In: Advances in clinical chemistry 2019 Jan. 1 (Vol. 93, pp. 63-113). Elsevier.
38. Sordillo P P, Sordillo L A, Helson L. Bifunctional role of pro-inflammatory cytokines after traumatic brain injury. Brain Inj. 2016 Jul. 28; 30(9):1043-53.
39. Koh Y. Update in acute respiratory distress syndrome. J Intensive Care Med. 2014 December; 2(1):1-6.
40. Boyle A J, Mac Sweeney R, McAuley D F. Pharmacological treatments in ARDS; a state-of-the-art update. BMC Med. 2013 Dec. 1; 11(1):166.
41. Patel V J, Biswas Roy S, Mehta H J, Joo M, Sadikot R T. Alternative and natural therapies for acute lung injury and acute respiratory distress syndrome. BioMed Res Int. 2018 May 16; 2018.
42. Villar J, Ferrando C, Martinez D, Ambrós A, Muñoz T, Soler J A, Aguilar G, Alba F, González-Higueras E, Conesa L A, Martin-Rodriguez C. Dexamethasone treatment for the acute respiratory distress syndrome: a multicentre, randomised controlled trial. Lancet Respir Med. 2020 Mar. 1; 8(3):267-76.
43. Zhang Z, Chen L, Ni H. The effectiveness of Corticosteroids on mortality in patients with acute respiratory distress syndrome or acute lung injury: a secondary analysis. Sci Rep. 2015 Dec. 2; 5:17654.
44. Schäcke H, Döcke W D, Asadullah K. Mechanisms involved in the side effects of glucocorticoids. Pharmacol Ther. 2002 Oct. 1; 96(1):23-43.

1. A method of treating a disease or pathology caused by an increase in levels of inflammatory cytokines comprising:
a compound of Formula I, 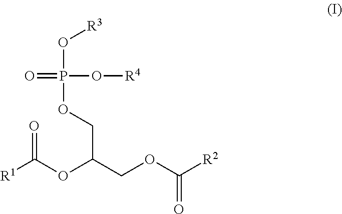
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 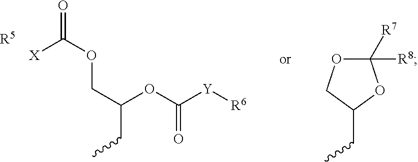
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁷ is a C₀-C₂₀ branched or unbranched hydrocarbon possessing 0-10 double bonds, 0-10 triple bonds or a combination of 0-10 double and triple bonds;
R⁸ is H or a C₀-C₂₀ branched or unbranched hydrocarbon possessing 0-10 double bonds, 0-10 triple bonds or a combination of 0-10 double and triple bonds;
X is a direct linkage, CH₂, O or NH;
Y is a direct linkage, CH₂, O or NH; and,
each stereogenic center is independently R, S or racemic.

2. The method of claim 1, wherein the disease or pathology caused by an increase in levels of inflammatory cytokines is a pulmonary inflammation, distress or insufficiency, bronchopulmonary dysplasia, asthma, chronic obstructive pulmonary disease, bronchitis, chronic or acute bronchoconstriction, acute respiratory distress syndrome, acute lung injury, cytokine storm, or bronchiectasis.

3. (canceled)

4. The method of claim 1, wherein R⁴ is H, Li, Na, K, Mg, Ca, Zn, Cs, ammonium or tetraalkylammonium.

5. The method of claim 1, wherein the compound is selected from at least one of:

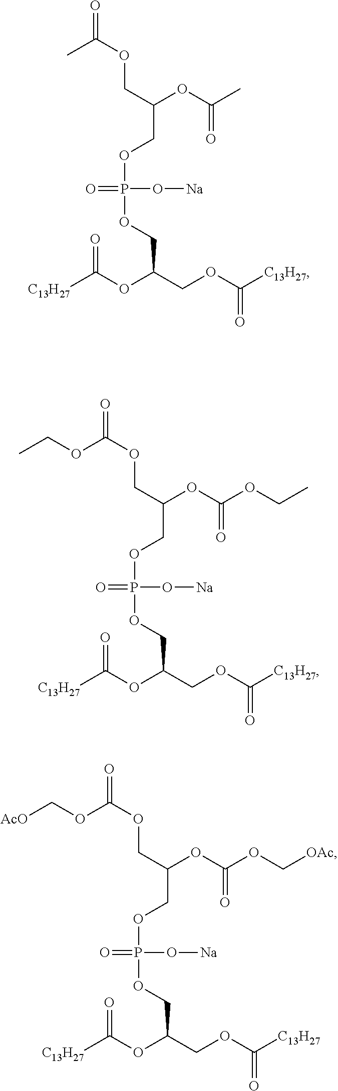

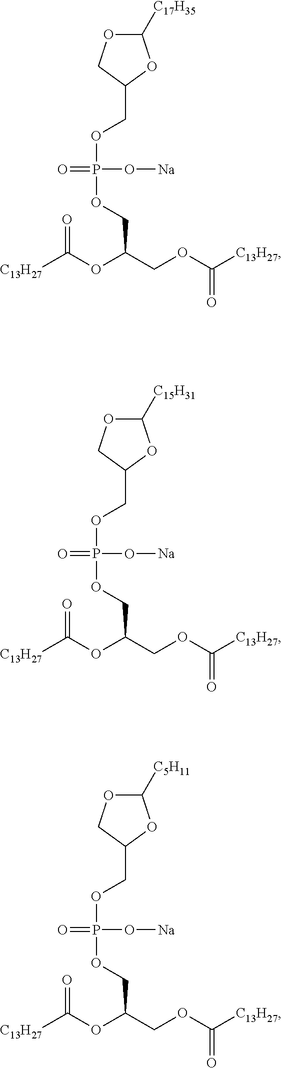

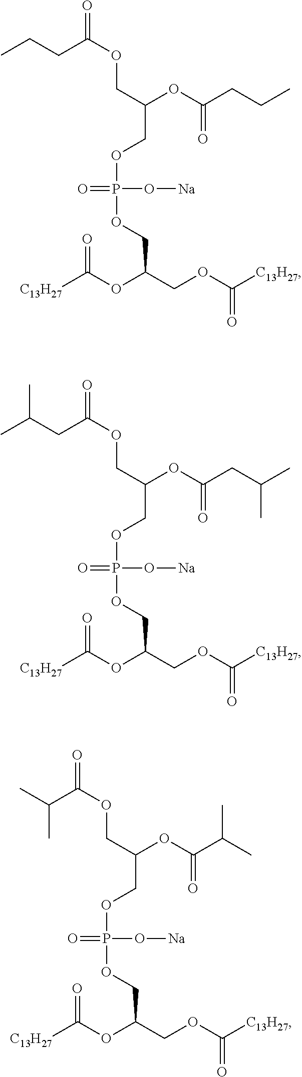

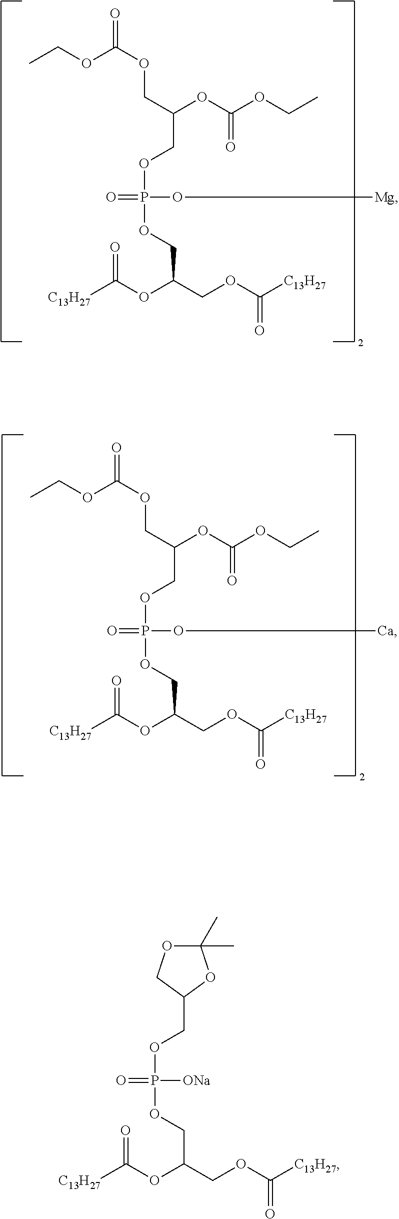

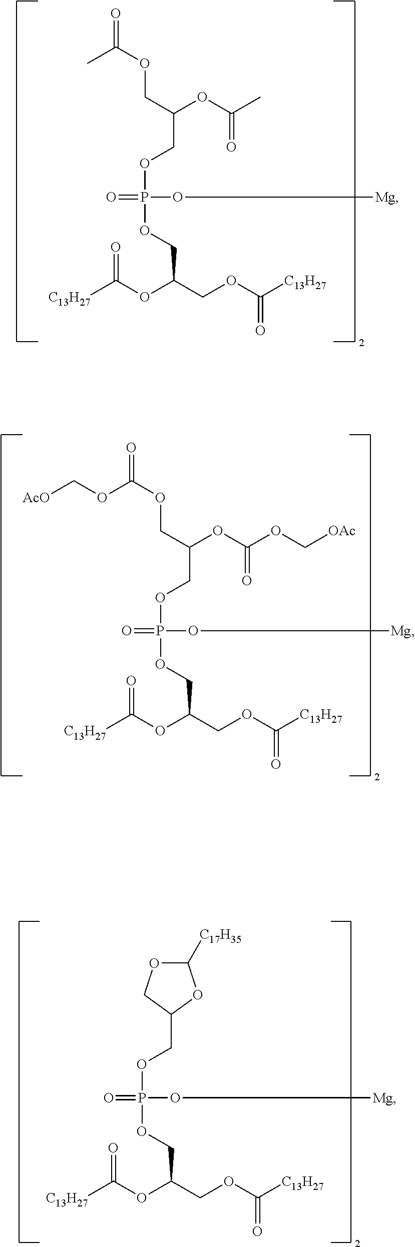

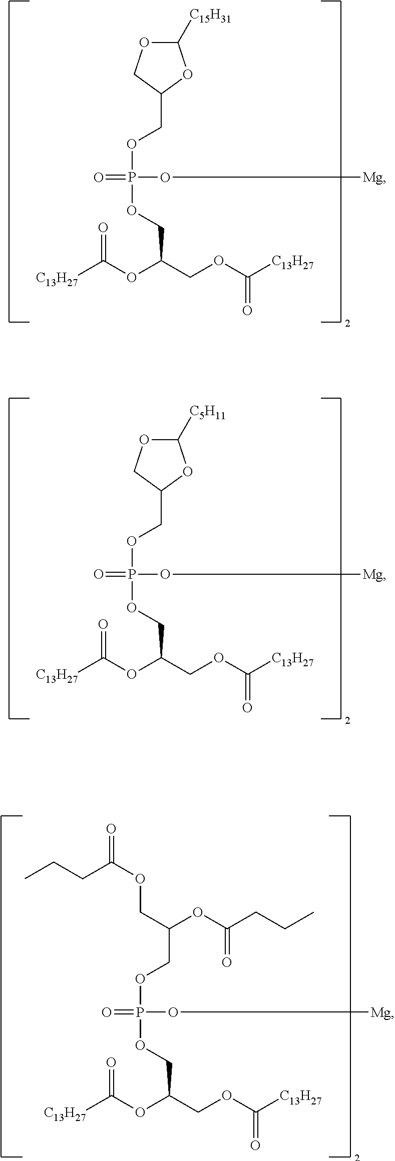

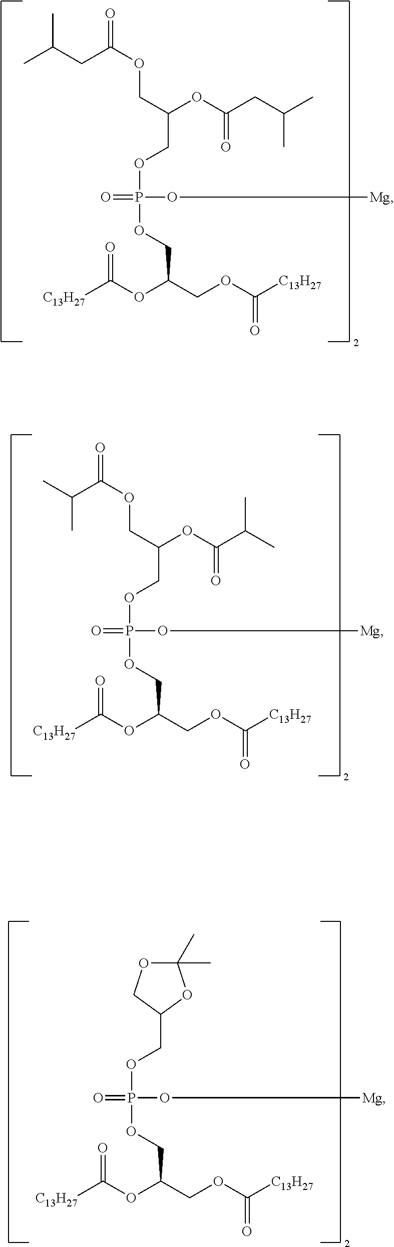

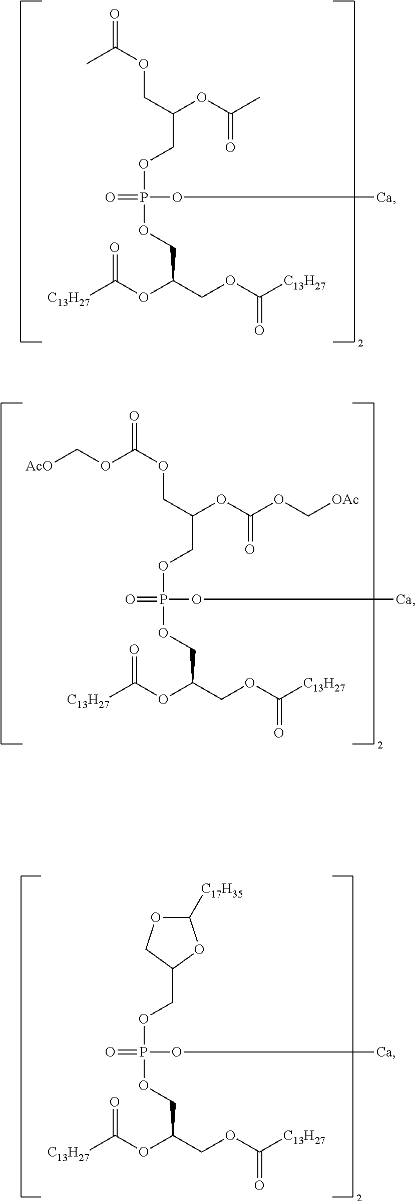

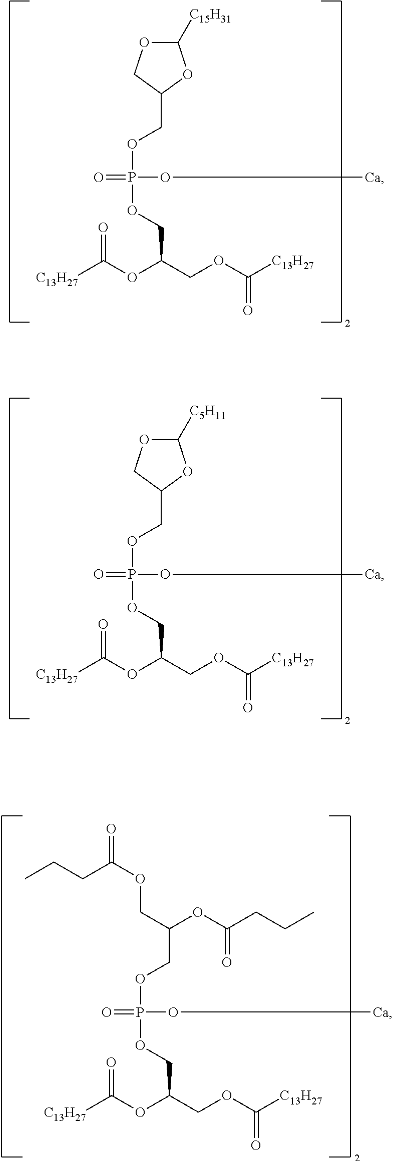

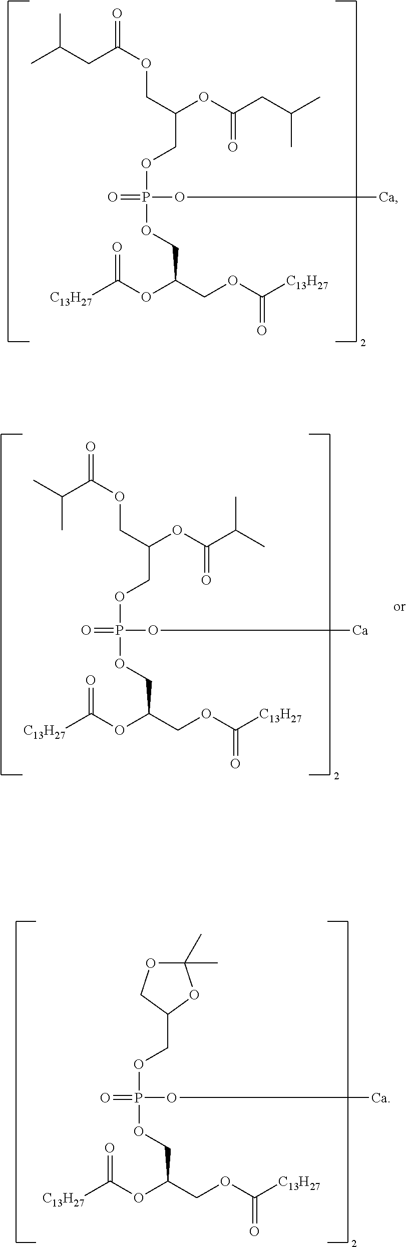

6. The method of claim 1, wherein at least one of:
the compound is a single entity, a solvate, a hydrate, a crystal, an amorphous solid, a liquid or an oil;
the compound is formulated for administration in at least once, once per day, twice per day, three times per day; or
the compound is administered at 0.1, 1, 2, 3, 4, 5, 6, 7, 89, 10, 15, 20, 25, 30, 40, 50, 60, 75, 80, 90, 100, 125, 150, 175, 200, 255, 250, 300, 400, or 500 mg/Kg.

7. (canceled)

8. (canceled)

9. The method of claim 1, wherein the composition is formulated into a pharmaceutical composition comprising one or more pharmaceutically acceptable excipients, buffers, one or more polymers, or salts, for oral, intravenous, nasal, pulmonary, alveolar, enteral, parenteral, topical aerosol, a nebulizer, or an inhaler administration.

10. (canceled)

11. (canceled)

12. (canceled)

13. The method of claim 1, further comprising an additional therapeutic agent selected from the group consisting of corticosteroids, bronchodilators, anticholinergics, vasodilators, diuretics, anti-hypertensive agents, acetazolamide, antibiotics, antivirals, immunosuppressive drugs, and surfactants.

14. (canceled)

15. The method of claim 1, wherein the compound is:

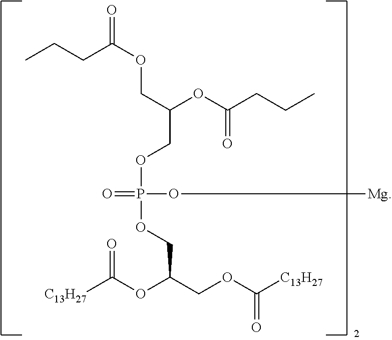

16. A method of treating a pulmonary inflammation, distress or insufficiency comprising:
a compound of Formula I, 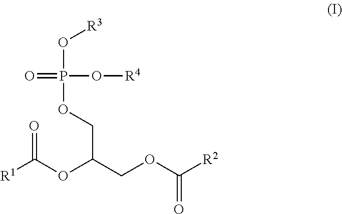
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 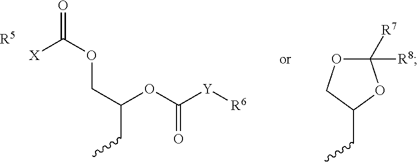
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁷ is a C₀-C₂₀ branched or unbranched hydrocarbon possessing 0-10 double bonds, 0-10 triple bonds or a combination of 0-10 double and triple bonds;
R⁸ is H or a C₀-C₂₀ branched or unbranched hydrocarbon possessing 0-10 double bonds, 0-10 triple bonds or a combination of 0-10 double and triple bonds;
X is a direct linkage, CH₂, O or NH;
Y is a direct linkage, CH₂, O or NH; and,
each stereogenic center is independently R, S or racemic.

17. The method of claim 16, wherein the pulmonary disease includes at least one of bronchopulmonary dysplasia, asthma, chronic obstructive pulmonary disease, bronchitis, chronic or acute bronchoconstriction, acute respiratory distress syndrome, acute lung injury, cytokine storm, or bronchiectasis.

18. The method of claim 16, wherein R⁴ is H, Li, Na, K, Mg, Ca, Zn, Cs, ammonium or tetraalkylammonium.

19. The method of claim 16, wherein the compound is selected from at least one of:

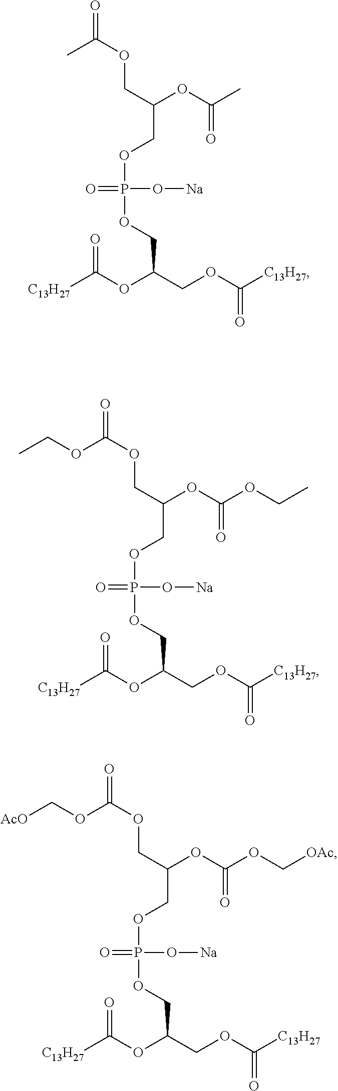

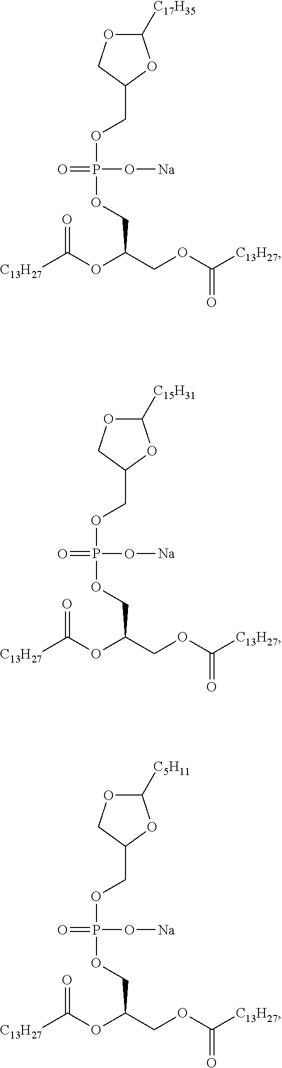

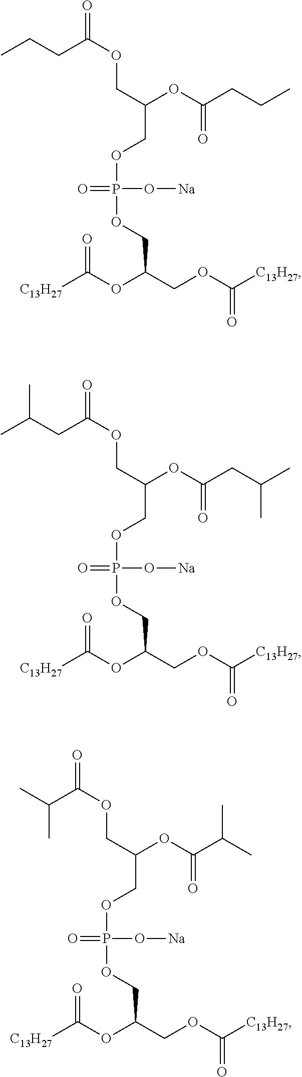

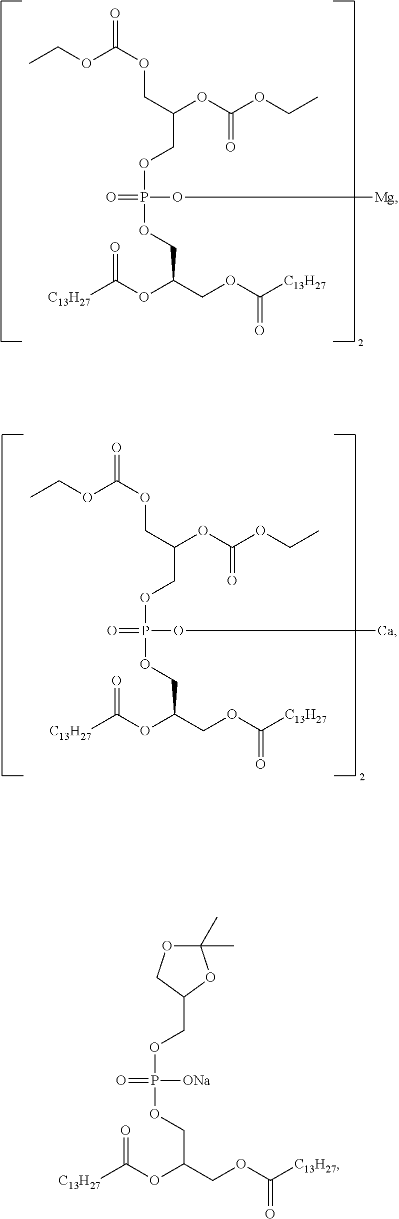

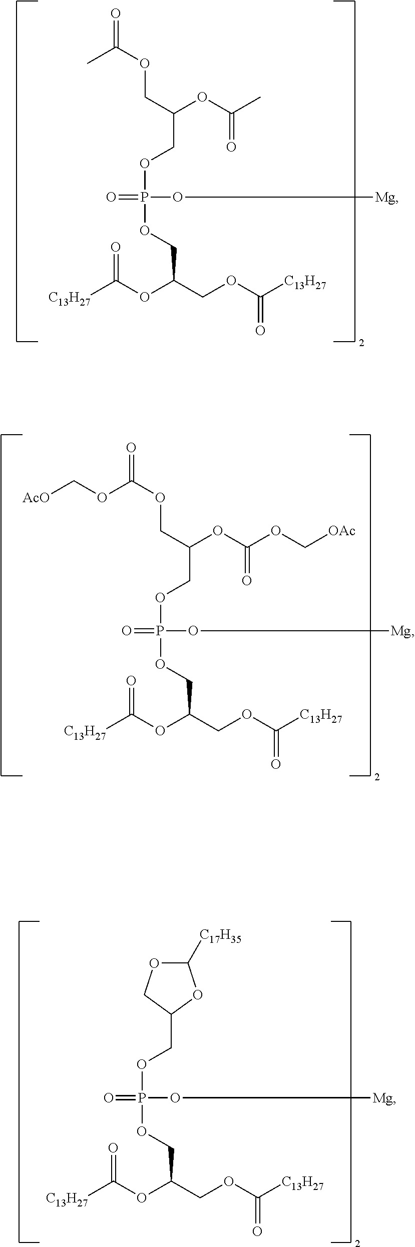

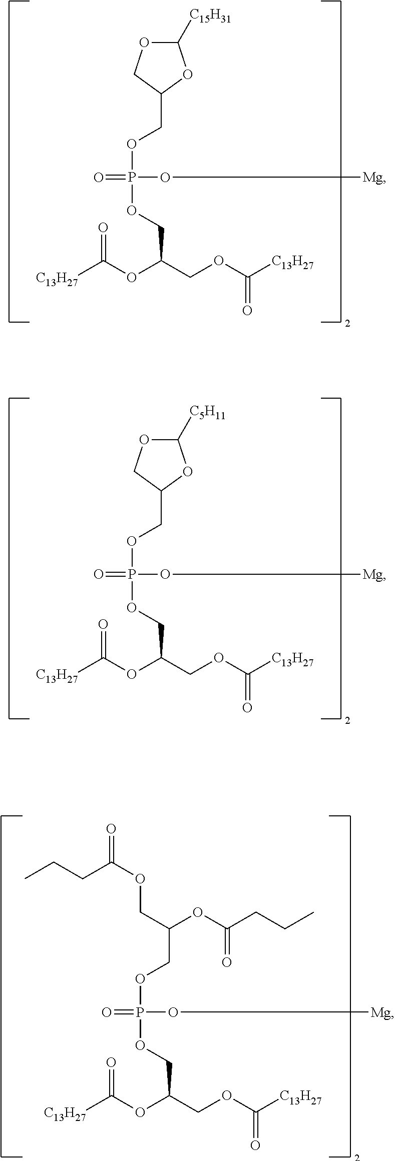

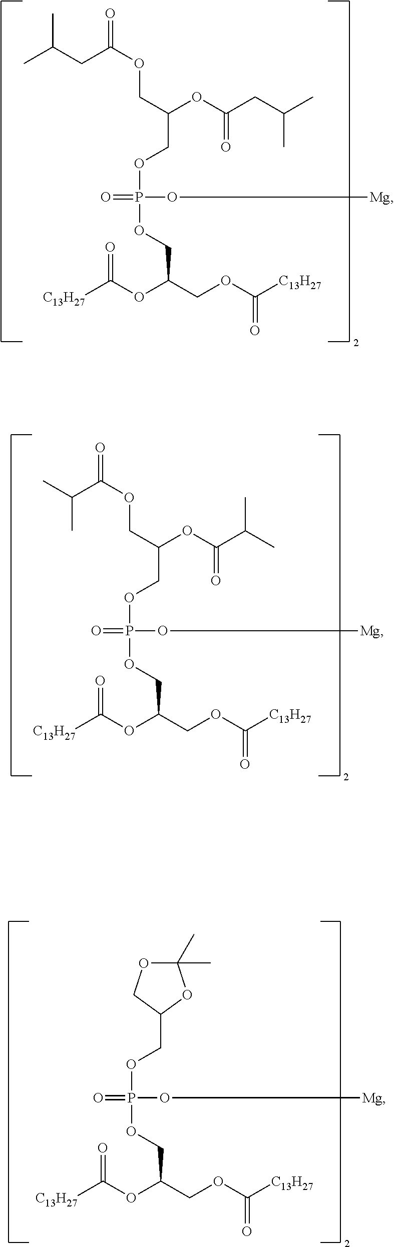

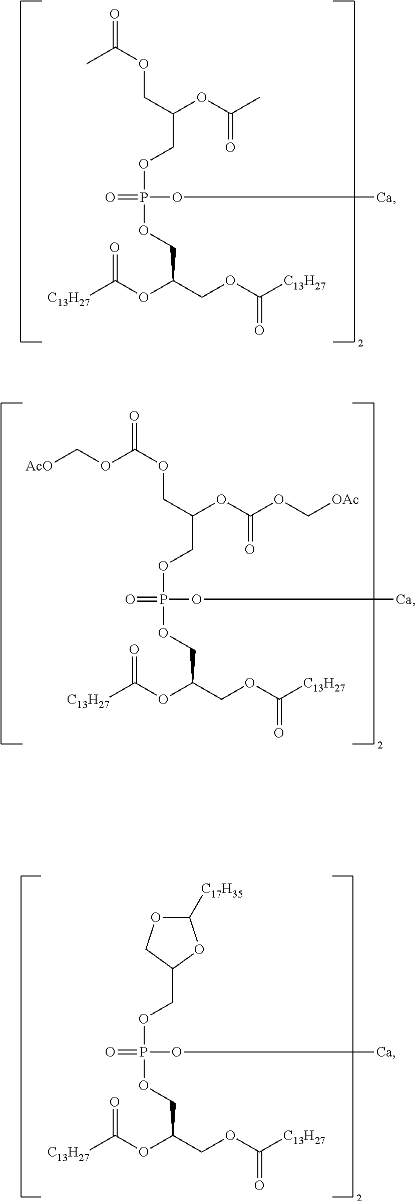

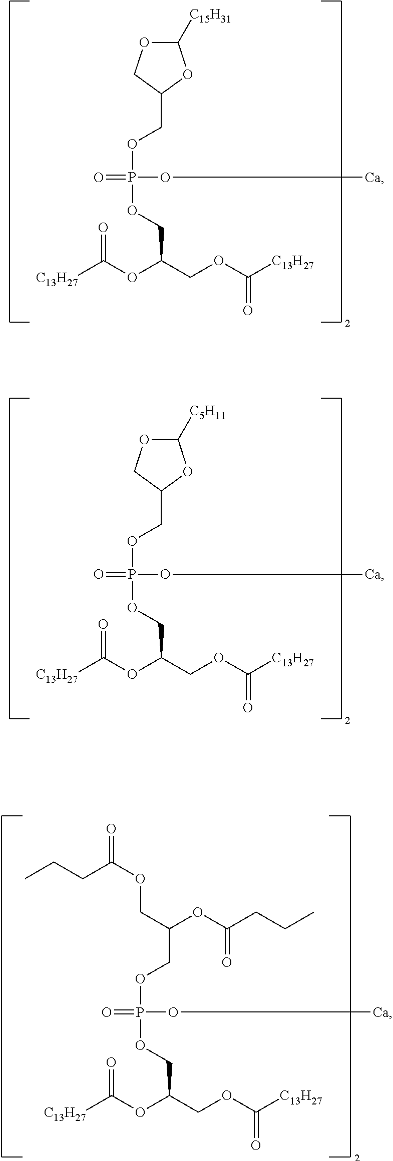

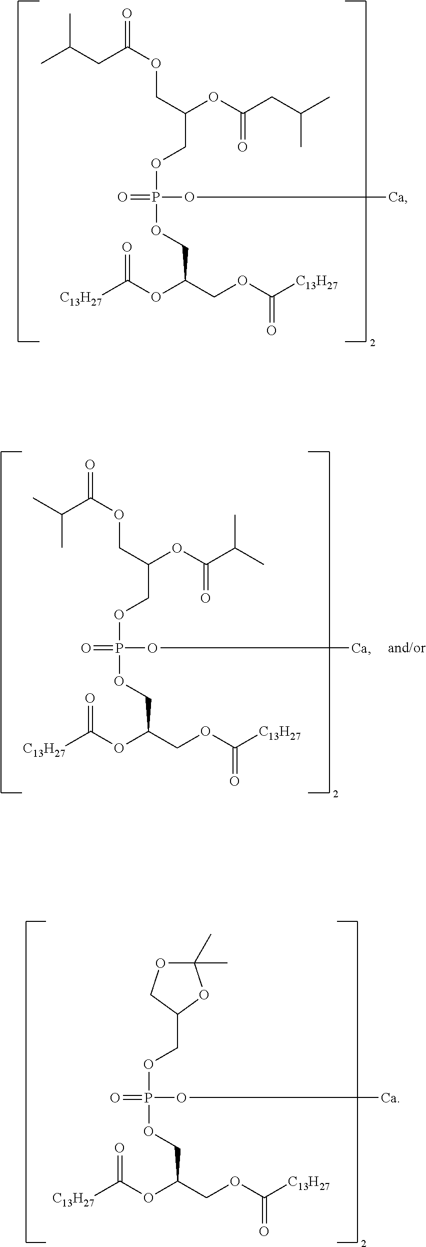

20. The method of claim 16, wherein at least one of:
the compound is a single entity, a solvate, a hydrate, a crystal, an amorphous solid, a liquid or an oil;
the compound is administered in at least once, once per day, twice per day, three times per day;
the compound is administered at 0.1, 1, 2, 3, 4, 5, 6, 7, 89, 10, 15, 20, 25, 30, 40, 50, 60, 75, 80, 90, 100, 125, 150, 175, 200, 255, 250, 300, 400, or 500 mg/Kg; or
the compound is formulated into a pharmaceutical composition adapted for oral, intravenous, nasal, pulmonary, alveolar, enteral, parenteral, or topical administration.

21. (canceled)

22. (canceled).

23. The method of claim 16, wherein the composition is formulated into a pharmaceutical composition comprising one or more pharmaceutically acceptable excipients, buffers, one or more polymers, or salts, for oral, intravenous, nasal, pulmonary, alveolar, enteral, parenteral, topical aerosol, a nebulizer, or an inhaler administration.

24. (canceled)

25. (canceled)

26. (canceled)

27. The method of claim 16, further comprising an additional therapeutic agent selected from the group consisting of corticosteroids, bronchodilators, anticholinergics, vasodilators, diuretics, anti-hypertensive agents, acetazolamide, antibiotics, antivirals, immunosuppressive drugs, and surfactants.

28. (canceled)

29. The method of claim 16, wherein the compound is:

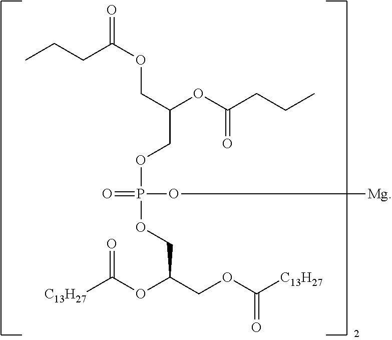

30. A method for preventing or treating a disease or pathology caused by an increase in levels of inflammatory cytokines comprising:
administering to the subject in need thereof a therapeutically effective amount of 1,2-dimyristoyl-sn-glycero-3-phosphocholine (DMPC), 1,2-Dimyristoyl-sn-glycero-3-phosphoglycerol (DMPG), or DMPC/DMPG, lysophosphatidylglycerol includes at least one of a lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine or a compound of formula (I) or stereoisomer, enantiomer, tautomer or a pharmaceutically acceptable salt thereof: 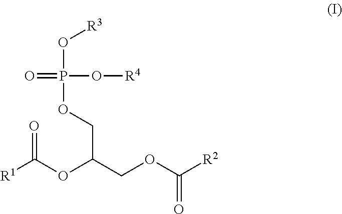
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 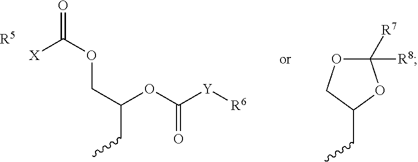
R⁴ is H or a pharmaceutically acceptable cation, wherein incorporation of said pharmaceutically acceptable cation results in a salt;
R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁷ is a C₀-C₂₀ branched or unbranched hydrocarbon possessing 0-10 double bonds, 0-10 triple bonds or a combination of 0-10 double and triple bonds;
R⁸ is H or a C₀-C₂₀ branched or unbranched hydrocarbon possessing 0-10 double bonds, 0-10 triple bonds or a combination of 0-10 double and triple bonds;
X is a direct linkage, CH₂, O or NH;
Y is a direct linkage, CH₂, O or NH; and,
each stereogenic center is independently R, S or racemic.

31. The method of claim 30, wherein the disease or pathology caused by an increase in levels of inflammatory cytokines is a pulmonary disease includes at least one of bronchopulmonary dysplasia, asthma, chronic obstructive pulmonary disease, bronchitis, chronic or acute bronchoconstriction, acute respiratory distress syndrome, acute lung injury, cytokine storm, or bronchiectasis.

32. (canceled)

33. The method of claim 30, wherein the compound is selected from at least one of:

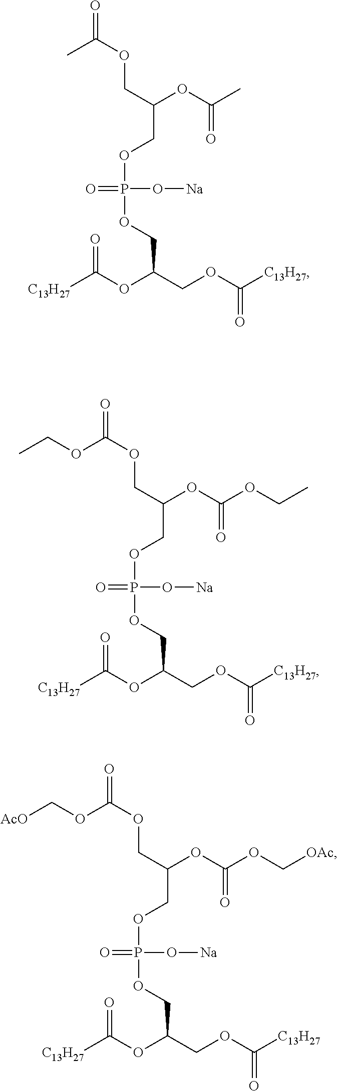

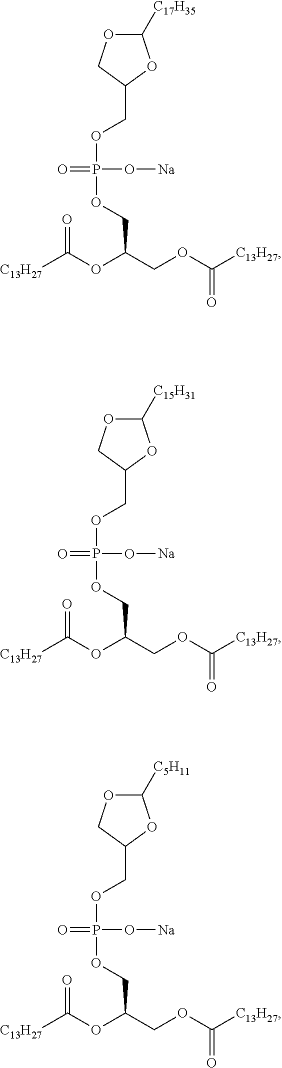

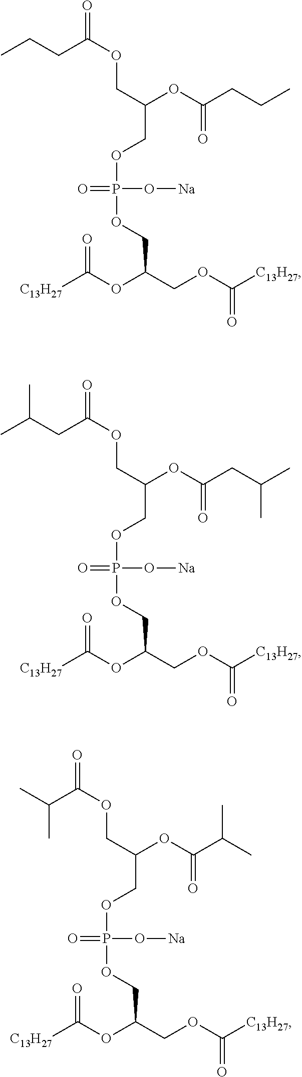

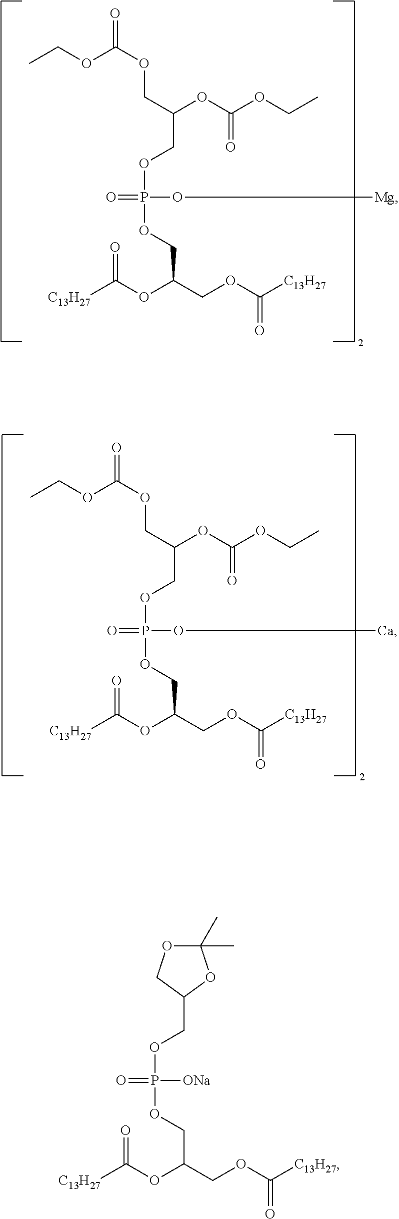

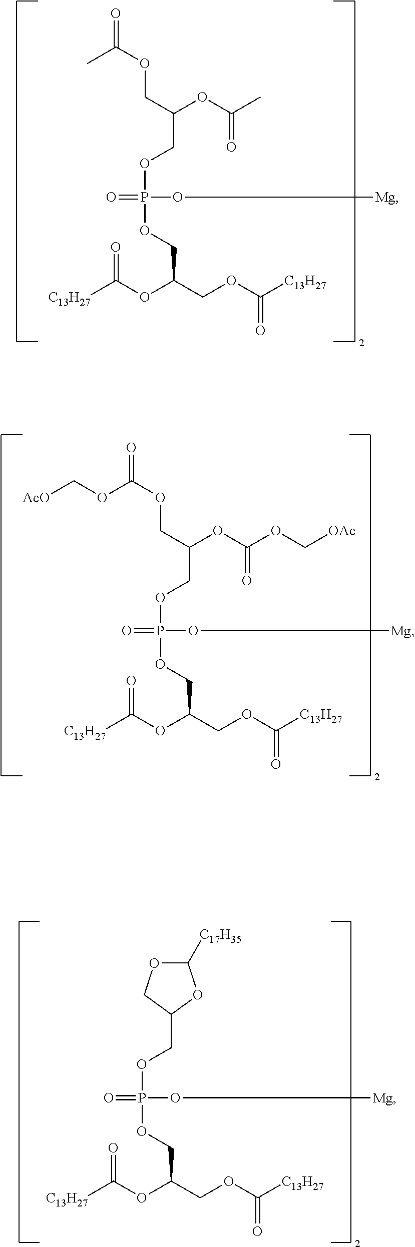

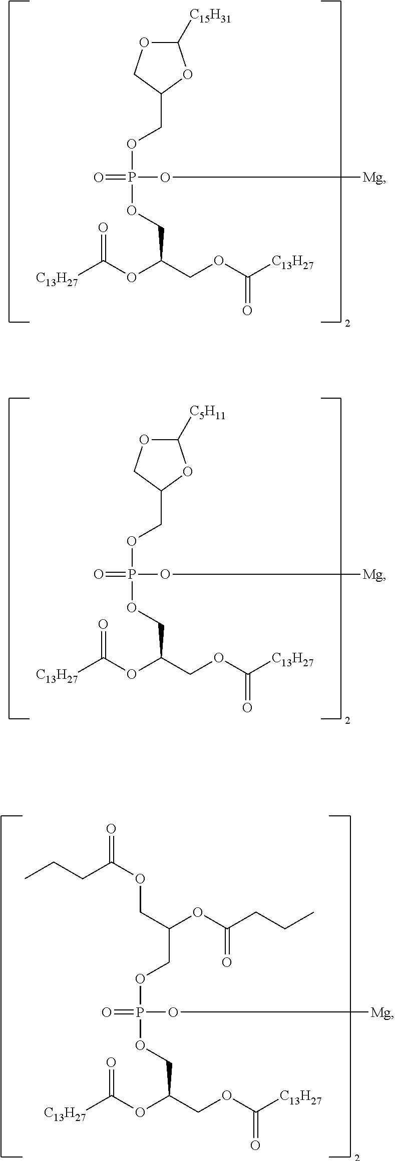

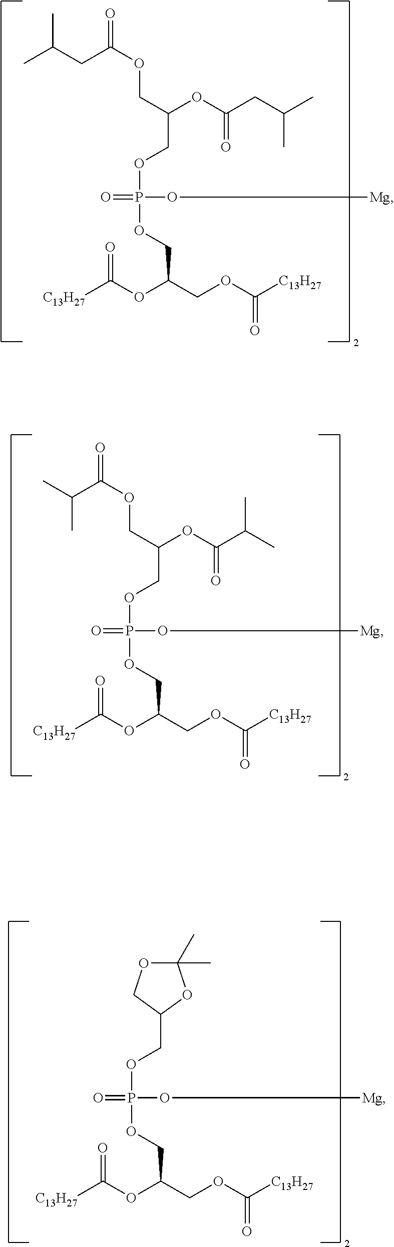

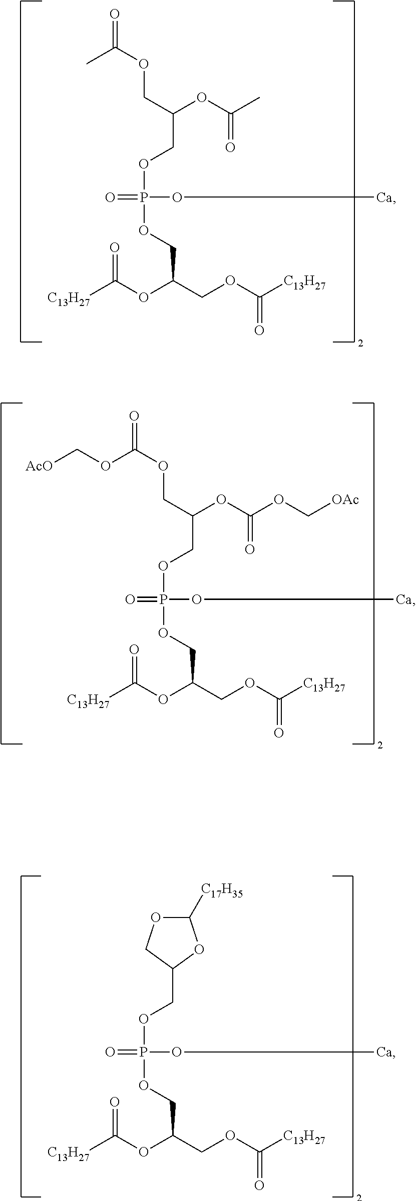

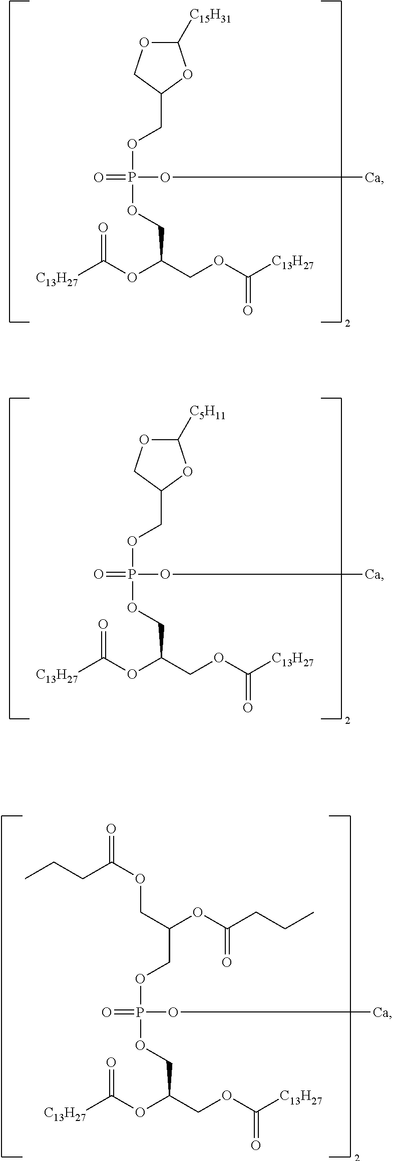

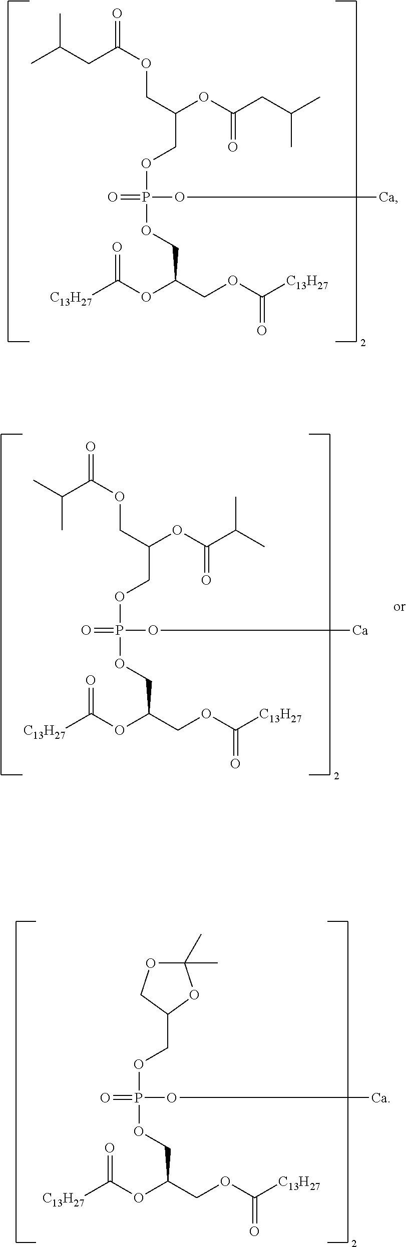

34. The method of claim 30, wherein at least one of:
the compound is a single entity, a solvate, a hydrate, a crystal, an amorphous solid, a liquid or an oil;
the compound is formulated for administration at least once, once per day, twice per day, three times per day; or
the compound is administered at 0.1, 1, 2, 3, 4, 5, 6, 7, 89, 10, 15, 20, 25, 30, 40, 50, 60, 75, 80, 90, 100, 125, 150, 175, 200, 255, 250, 300, 400, or 500 mg/Kg.

35. (canceled)

36. (canceled)

37. The method of claim 30, wherein the composition is formulated into a pharmaceutical composition comprising one or more pharmaceutically acceptable excipients, buffers, one or more polymers, or salts, for oral, intravenous, nasal, pulmonary, alveolar, enteral, parenteral, topical aerosol, a nebulizer, or an inhaler administration.

38. (canceled)

39. (canceled)

40. (canceled)

41. The method of claim 30, further comprising an additional therapeutic agent selected from the group consisting of corticosteroids, bronchodilators, anticholinergics, vasodilators, diuretics, anti-hypertensive agents, acetazolamide, antibiotics, antivirals, immunosuppressive drugs, and surfactants.

42. (canceled)

43. The method of claim 30, wherein the compound is:

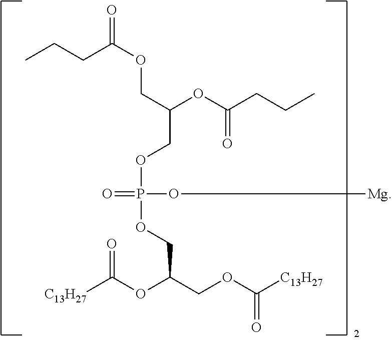

44. The method of claim 30, further comprising the step of identifying a subject in need of treatment for a pulmonary inflammation, distress or insufficiency prior to the treatment.

45. The method of claim 30, wherein the compound is:

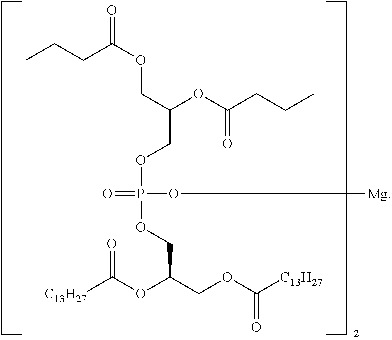

46. The method of claim 30, further comprising the step of identifying a subject in need of treatment for a pulmonary inflammation, distress or insufficiency prior to the treatment.
